(19) United States
(12) Patent Application Publication
(10) Pub. No.: US 2023/0200725 A1
(43) Pub. Date: Jun. 29, 2023

(54) Method and Apparatus for Non-Invasive Detection of Pathogens in Wounds

(71) Applicant: Tao Treasures LLC DBA Nanobiofab, Frederick, MD (US)

(72) Inventor: Xiaonao LIU, Frederick, MD (US)

(21) Appl. No.: 18/179,604

(22) Filed: Mar. 7, 2023

Related U.S. Application Data
(63) Continuation-in-part of application No. 17/751,207, filed on May 23, 2022.
(60) Provisional application No. 63/192,005, filed on May 22, 2021.
(60) Provisional application No. 63/192,006, filed on May 22, 2021.
(60) Provisional application No. 63/269,151, filed on Mar. 10, 2022.
(60) Provisional application No. 63/269,151, filed on Mar. 10, 2022.
(60) Provisional application No. 63/434,064, filed on Dec. 20, 2022.

(51) Int. Cl. A61B 5/00

(52) U.S. Cl. CPC A61B 5/445; A61B 2010/0083

(57) ABSTRACT

According to a present invention embodiment, at least one sensor detects one or more gases emanating from one or more pathogens in a wound that produce an infection. The at least one sensor includes sensing materials that change one or more properties in response to a presence of the one or more gases. At least one processor analyzes information from the at least one sensor to identify the one or more pathogens and determine a presence of the infection in the wound. The one or more pathogens are identified based on patterns of changes of the one or more properties indicating corresponding pathogens. The at least one sensor may be disposed within one of a wearable device, a portable device, and a wound dressing. In addition, a negative pressure source may be utilized to apply negative pressure to the wound to promote healing.

### CROSS REFERENCE TO RELATED APPLICATIONS

This application is a Continuation-in-part of U.S. patent application Ser. No. 17/751,207, entitled “Noninvasive Device for Monitor, Detection, and Diagnosis of Diseases and Human Performance” and filed May 23, 2022, which claims priority to U.S. Provisional Patent Application Ser. No. 63/192,005, entitled “Noninvasive Wearable Intelligent Sensor for Rapid Monitoring, Screening, and Diagnosis of Diseases from Skin” and filed May 22, 2021, U.S. Provisional Patent Application Ser. No. 63/192,006, entitled “Noninvasive Wearable/Portable Intelligent Sensor for Rapid Monitoring, Screening, and Diagnosis of Diseases from Skin” and filed May 22, 2021, and U.S. Provisional Patent Application Ser. No. 63/269,151, entitled “Method and Device for Non-Invasive Detecting and Identifying Pathogen in Real-Time in Wounds” and filed Mar. 10, 2022. The disclosures of the above-identified patent applications are hereby incorporated by reference in their entireties.

This application also claims priority to U.S. Provisional Patent Application Ser. No. 63/269,151, entitled “Method and Device for Non-Invasive Detecting and Identifying Pathogen in Real-Time in Wounds” and filed Mar. 10, 2022, and U.S. Provisional Patent Application Ser. No. 63/434,064, entitled “Integrated Sensor in Negative Pressure Wound Device for Wound Monitoring and Early Detection of Infection” and filed Dec. 20, 2022, the disclosures of which are hereby incorporated by reference in their entireties.

### TECHNICAL FIELD

Present invention embodiments pertain to detecting and identifying pathogens for early detection of wound infection in real-time.

### BACKGROUND

### Discussion of Related Art

Chronic cutaneous wound infections and surgical site infections present a huge burden on the healthcare system in the United States and can lead to increased morbidity and mortality. Common pathogens associated with chronic as well as superficial and deep surgical site infections include, but are not limited to, Staphylococcus epidermidis (SE), Streptococcus pyogenes (SP), Enterococcus faecium (EF), Staphylococcus aureus (SA), Klebsiella pneumonia (KP), Acinetobacter baumannii (AB), Pseudomons aeruginosa (PA), Enterobacter species (ES), Escherichia coli (EC), Proteus mirabilis (PM), Serratia marcescens (SM), Enterobacter clocae (E.cl), and Acetinobacter anitratus (AA).

Negative pressure wound devices (NPWDs) are commonly used in the treatment of wounds, as they help to remove excess fluid and promote healing. However, wounds can become infected, which can significantly delay healing and can lead to serious complications if not properly treated. Early detection of wound infections is critical for ensuring timely treatment and optimal patient outcomes.

Current diagnostic methods of identifying and confirming infection involve visual inspection, and culture-based and molecular methods. These techniques are time and resource consuming and some require sample transport. Many also possess limited sensitivity and specificity inherent to sample processing and user error (requiring complex laboratory science experience and equipment).

Thus, the limitations of these approaches can delay diagnostics, often resulting in empirical treatment before confirmation of the infectious agent, increasing the risk for sub-optimal choice of antibiotics. This often contributes to the development of antibiotic resistance and an increase in mortality. In addition, these approaches may not provide real-time results, making it difficult to promptly detect and treat infections.

Volatile organic compounds (VOCs) as a diagnostic tool include a diverse group of carbon-based molecules, including alcohols, isocyanates, ketones, aldehydes, hydrocarbons and sulphides, which are volatile at ambient temperatures. VOC detection has the advantage of being painless, non-invasive and reproducible. There is increasing evidence that VOCs and combinations thereof are unique to various disease states and their early detection could represent a useful means of diagnosis. VOCs have been identified as potential biomarkers in diagnosis of lung cancer, breast cancer, asthma, and diabetes.

Pathogens also produce VOCs, and currently volatile detection via breath testing has been at the forefront of this technology to diagnose infection. The ability to rapidly detect microbial VOCs, potentially allowing identification of pathogens, has immense implications in the management of infection, from triage, point-of-injury care in austere environments to hospitals, in the clinic and home setting as well. If a patient's wounds can be accurately monitored from early stage, to discharge from the hospital, to in home use, then appropriate antimicrobial therapy can be initiated early enough to prevent a more serious infection, and the status of the infection could be monitored continuously.

### SUMMARY

According to one embodiment of the present invention, a system detects a wound infection. The system comprises at least one sensor and at least one processor. The at least one sensor detects one or more gases emanating from one or more pathogens in a wound that produce an infection. The at least one sensor includes sensing materials that change one or more properties in response to a presence of the one or more gases. The at least one processor analyzes information from the at least one sensor to identify the one or more pathogens and determine a presence of the infection in the wound. The one or more pathogens are identified based on patterns of changes of the one or more properties indicating corresponding pathogens. In an embodiment, the at least one sensor is disposed within one of a wearable device, a portable device, and a wound dressing. In an embodiment, the system further comprises a negative pressure source to apply negative pressure to the wound to promote healing. Embodiments of the present invention further include a method and an apparatus with a memory device containing software executable by at least one processor to detect a wound infection in substantially the same manner described above.

### BRIEF DESCRIPTION OF THE DRAWINGS

Generally, like reference numerals in the various figures are utilized to designate like components.

FIG. 1 illustrates an exemplary wearable device according to an embodiment of the present invention.

FIG. 2 is a diagram showing components in a device of an embodiment of the present invention.

FIG. 3 shows a flow chart of an example method for preparing sensing materials of sensors in a sensor array for use with embodiments of the present invention.

FIG. 4 shows TEM images and response of VOC gases of sensing materials in an example sensor array.

FIG. 5 shows a process for building a model/classifier and using the model/classifier to diagnose wound infections according to an embodiment of the present invention.

FIG. 6A illustrates a portable device according to an embodiment of the present invention.

FIG. 6B illustrates use of the portable device of FIG. 6A for detecting infections in wounds according to an embodiment of the present invention.

FIGS. 7A and 7B show an example analysis of VOCs patterns of bacteria including Escherichia coli (E. coli), Pseudomonas aeruginosa (PA), and Staphylococcus aureus (SA) in a wound infection using the device of FIG. 6A.

FIG. 8 illustrates a wearable device integrated into a dressing system for real-time monitoring of wound infection according to an embodiment of the present invention.

FIGS. 9A-9D show example VOC patterns of pathogens due to wound infection detected by the wearable device of FIG. 8.

FIG. 10A shows sensor measurements of analytes from an example embodiment of the present invention.

FIG. 10B shows sensor data from an example embodiment of the present invention projected into a set of principal components.

FIG. 10C shows sensor data from an example embodiment of the present invention projected into a different set of principal components.

FIG. 10D shows accuracy of prediction for bacteria of an example embodiment.

FIG. 11 shows another example wearable device for detecting wound infection according to an embodiment of the present invention.

FIG. 12A illustrates a negative pressure wound device according to an embodiment of the present invention.

FIG. 12B illustrates use of the negative pressure wound device of FIG. 12A according to an embodiment of the present invention.

### DETAILED DESCRIPTION OF EXAMPLE EMBODIMENTS

An embodiment of the present invention provides real-time detection of the presence or absence in a subject's wound of an infection, and identification of the presence of one or more particular pathogens in the wound.

A present invention embodiment is directed to a wearable or portable device or a system comprising a sensor array with a plurality of sensors, a detection mechanism and a pattern recognition analyzer and use thereof for diagnosing the wound infection in a non-invasive real-time manner.

A device of a present invention embodiment may be embedded in a wound dressing or in proximity to the wound. The device is comprised of a MACchip sensor array module, micro-controller unit (MCU) modules, digital signal processing circuit (DSC), analog-to-digital converters (ADC), communication interfaces (USB, Bluetooth, and WiFi), “on/off switch”, and user interface. The sensor array module comprises multiple-component nanostructured material-based sensors or a plurality of sensors in conjunction with a pattern recognition and machine learning or other algorithm. The device may be utilized to provide convenient, non-invasive, real-time detection of all stages of infection, from early onset of wound infection and thereafter, thereby enabling caregivers to provide effective and timely treatment.

Present invention embodiments detect the development of infection directly on the wound bed and provide a simultaneous identification of the active microorganism for wound infection management in the hospital or other settings. Present invention embodiments provide a noninvasive technique utilizing a nanomaterial-based sensor that can detect early stages of infection before symptoms develop and enable consistent monitoring through all phases of infection.

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in embodiments of the invention. It is to be understood that the value to which the modifier “about” or “approximately” refers is itself also specifically, and preferably, disclosed.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

The term “real-time” is used to describe a process of sensing, processing, or transmitting information in a time frame which is equal to or shorter than the minimum timescale at which the information is needed. For example, the real-time monitoring of pulse rate may result in a single average pulse-rate measurement every minute, averaged over 30 seconds, because an instantaneous pulse rate is often useless to the end user. Typically, averaged physiological and environmental information is more relevant than instantaneous changes. Thus, in the context of some embodiments of the present invention, signals may sometimes be processed over several seconds, or even minutes, in order to generate a “real-time” response.

The terms “infection” and “bacterial infection” indicate the presence and/or colonization of pathogenic bacteria in or on a subject in a number or an amount sufficient to be pathogenic, that is sufficient to cause disease, damage or harm to a subject infected with said bacterium. A subject having an infection is said to be “infected” with a pathogen. Pathogenic bacteria or short “pathogens” as used herein are bacteria that are known to cause bacterial infections in subjects.

The terms “comprises”, “comprising”, “include”, “including”, and the like are used to specify the presence of stated elements, steps, operations, and/or components, but do not preclude the presence or addition of one or more other elements, steps, operations, and/or components. The terms “first,” “second,” and the like may be used to describe various elements, but do not limit the elements. Such terms are only used to distinguish one element from another.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Present invention embodiments may be used for detecting and identifying wound infections using VOCs released from the pathogens of a subject (e.g., in the skin of palm, finger, ear, nose, face, eye, arm, leg, chest, breast, back, abdomen, and/or or foot), thus allowing real-time monitoring of the dynamic change of VOCs. Smart nanosensors in conjugation with pattern recognition and machine-learning algorithms enable early detection of wound infection.

A device of an embodiment of the present invention may be used for the detection and identification of pathogens in the subject in real-time. The device comprises of at least one sensor array, a micro-controller unit (MCU), a digital signal processing circuit (DSC), analog-to-digital converters (ADC), communication interfaces (USB, Bluetooth, and WiFi), and an “on-off switch”. Results are transferred via wireless communication in real-time to a cellphone or laptop, and/or to a designated server for data analysis and storage, with a user interface. Information includes vital signs (such as skin temperature), VOCs information, and environment condition (time, temperature, humidity, and/or pressure). Based on collected information, a comprehensive informational library may be built to support pattern recognition and machine learning algorithms for early detection of wound infection.

In an embodiment, a method or process for diagnosing wound infection comprises: applying the device on or in proximity of the wound, such as attaching or embedding the device into a wound dressing system, wound healing system, or wound management system; detecting metabolite VOC gases formed therefrom emanating from the wound in real-time using a nanostructured sensor array; analyzing electrical characteristics in response thereto; and recognizing and identifying pathogens using pattern recognition and machine learning algorithms. In addition, the method may further comprise diagnosing the infection and/or identification of bacteria in one of internal medicine, rheumatology, physical medicine, rehabilitation, clinical research, and basic research in the fields of immunology and/or microbiology; and evaluating the efficacy of a drug to the subject that is known to kill or inhibit the growth of the bacteria causing the infection.

An embodiment of the present invention provides a wearable and/or portable device for rapid screening and diagnosis of pathogens in vivo and in vitro. The device comprises a housing having an opening structure, and at least one open end of the device disposed on the housing having nanosensors and/or biosensors to detect the VOCs emanated from the pathogen for data acquisition. Data is provided to a remote server connected to nanosensors and/or biosensors for acquiring data in the device of the housing for processing and sent to an acquisition unit.

In some embodiments, the device comprises a sensor array module for VOCs detection, sensors for monitoring vital signs (e.g., heart rate, blood pressure, respiratory rate, blood oxygen saturation, and/or skin and/or body temperature), Artificial Intelligence/machine learning algorithms, and an intuitive, user-friendly interface.

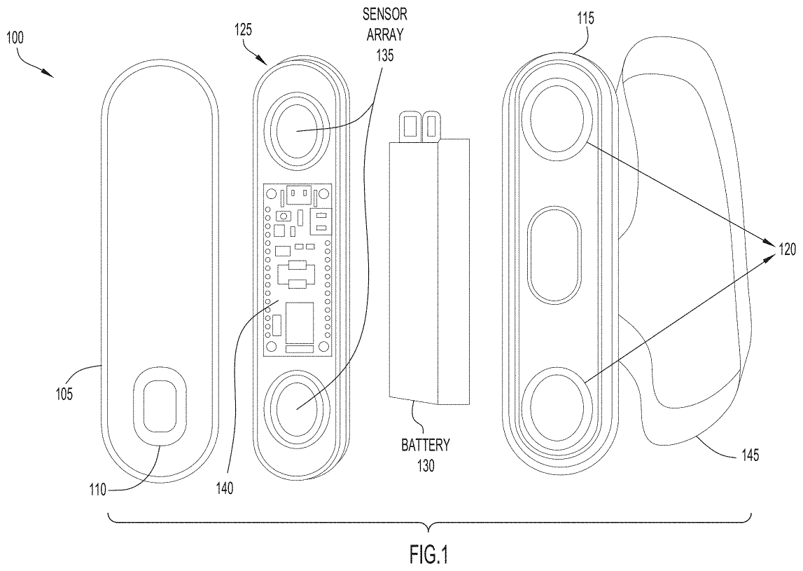

FIG. 1 shows an example of a device 100 that may be worn by a user or subject according to an embodiment of the present invention. Device 100 includes a top cover 105 having indicators 110, such as OLED (or an LCD display which a user may interact with), showing the status of the device, a bottom cover 115 having two cup-shaped inlets 120 adapted to be attached to the surface of a test subject, as well as electronics 125 and a battery 130 housed between the top and the bottom cover. The cup-shaped inlets are adapted to allow VOCs emanated from the wound of the test subject to enter the device. The electronics includes two sensor arrays 135, each having multiple sensors for detecting VOCs, a chip containing electronics for recording (e.g., in a non-volatile memory), processing (e.g., by a processor), and transmitting (e.g., through Bluetooth card 140, Wifi card, etc.) data. The battery powers the electronics. In this embodiment, a cup-shaped inlet on the bottom cover is connected with a sensor array in the electronics through a conduit (e.g., a tube) so that the sensor array is exposed to VOCs entering the inlet immediately. The device has a strap 145 to affix it to a limb or the torso of a human or a mammal subject (e.g., to the palm, finger, ear, nose, face, eye, arm, leg, chest, breast, back, abdomen, and/or or foot).

In certain embodiments, the inlet on the device is covered by a membrane that is waterproof and/or breathable. The sensor array contains multiple VOC sensors that react to VOCs and produce signals when exposed to gases, vapors, or odor containing VOCs released from pathogens of a wound of a subject as well as one or more physiological sensors. The device can integrate with multiple commercially available physiological sensors for monitoring vital signals. Such vital signals may include heart rate, pulse rate, respiratory rate, blood oxygen saturation, blood pressure, hydration level, stress, position and balance, body strain, neurological function, brain activity, blood pressure, cranial pressure, auscultatory information, skin and body temperature, sleep, cholesterol, lipids, blood panel, body fat density, and/or muscle density. Additional sensors may be installed that monitor environment conditions, such as temperature, humidity, and/or pressure. The collected data can be used for pattern recognition and machine learning algorithms, which can be used for detection of wound infection and perform other functions.

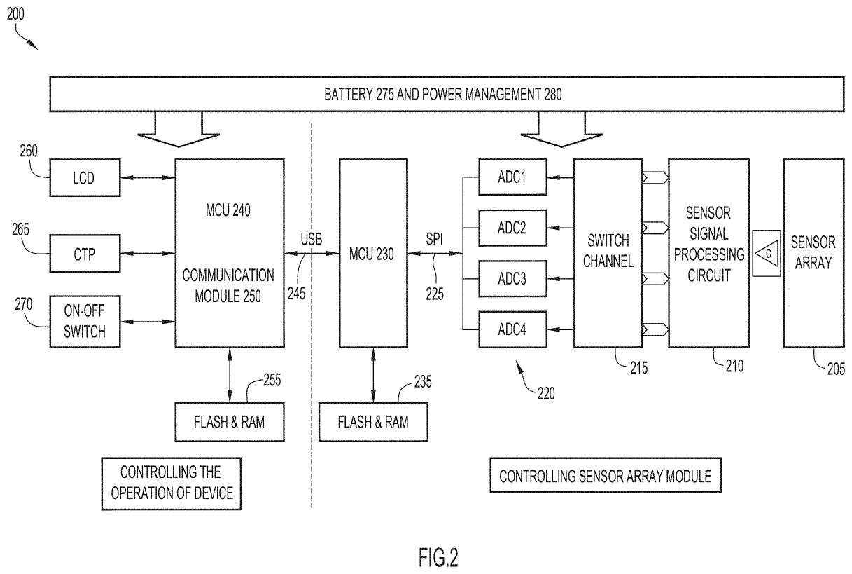

FIG. 2 is a block diagram illustrating various components of a device 200 for detecting wound infection according to an embodiment of the present invention. Device 200 may correspond to any of the devices described herein (e.g., for FIGS. 1, 6A, 6B, 8, 11, 12A, and 12B). The device includes sensor array 205 that contains a plurality of VOC sensors and, optionally, one or more physiological sensors and/or environment sensors. The plurality of sensor signals are sent to a sensor signal processing circuit 210 to be processed. A switch channel 215 selects signals from one of the sensors at a time and sends it to one of the analog-to-digital converters (ADC) 220 to convert it to digital signals. The digital signals pass through the serial peripheral interface (SPI) 225 into a micro-controller Unit (MCU) 230. The MCU 230 may be connected to a Flash memory or a RAM 235 for data storage and/or retrieval. In the embodiment of FIG. 2, MCU 230 is connected to an MCU 240 through a USB interface 245. MCU 240 is a part of a communication module 250, which transfers the processed output to proximate devices via a communication device, e.g., a Bluetooth card or a WiFi card. The communication module 250 is further connected to its own Flash/RAM memory 255, as well as a user interface such as a liquid crystal display (LCD) 260, a capacitive touch panel (CTP) 265, and an on-off switch 270.

A battery 275 and a power management circuit 280 control the power supply to the sensor array and other electronics. Note that a user can control the sensor array 205 by sending commands through the user interface. The device may also have embedded firmware that runs the device.

In certain embodiments, raw data detected by sensor array 205 or processed data is transferred wirelessly in real-time to a cellphone or laptop, and/or to a designated server for data analysis and storage. The transferred data may include vital signs (such as heart rate, blood pressure, respiratory rate, blood oxygen saturation, and/or skin and/or body temperature), VOCs information, and environment condition (time, temperature, humidity, and/or pressure). Based on collected information, a comprehensive database can be built to support the pattern recognition and machine learning algorithms for early detection of wound infection.

In some embodiments, the device measures the VOCs based on a nanostructured sensor array. The sensor array comprises a plurality of sensors, for example between 2 and 6 and 8 and 12 and 32 sensors or more with sensing materials, each sensor containing a material that changes certain properties, e.g., resistance, when contacting certain VOCs. The sensing material comprises at least one or two or more or mixture nanoporous structure, like mesoporous, macroporous, microporous, nanoporous, non-porous, hierarchical porous materials, including mesoporous/macroporous hierarchical structure, microporous/macroporous hierarchical structure, microporous/mesoporous hierarchical structure, microporous/mesoporous/macroporous hierarchical structure, etc. The mesoporous structure is in a configuration selected from a well-ordered mesoporous structure with regular pore arrangement, a worm-like mesoporous structure with uniform pore size but without long-range regularity, or a non-order mesostructure with pore size from 2-50 nm. The macroporous structure is in a configuration selected from a well-ordered macroporous structure or non-order macroporous structure with pore size from 50 nm to 50 μm. In a further embodiment, the pore size of sensing materials is ranging from 0.4 to 2 nm, 2-50 nm, 50 nm to 200 nm, 200 nm to 500 nm, 500 nm to 1 μm, 1-50 μm. The specific surface area is 1-1000 m²/g.

In some embodiments, the sensing material comprises unary, binary, ternary, quaternary, quinary, senary, septenary, and octonary multiple-component metal oxides, selected from an element group of tin (Sn), terbium (Tb), cobalt (Co), zinc (Zn), indium (In), copper (Cu), nickel (Ni), chromium (Cr), manganese (Mn), tungsten (W), titanium (Ti), vanadium (V), iron (Fe), aluminum (Al), gallium (Ga), silver (Ag), gold (Au), palladium (Pd), rhodium (Rh), ruthenium (Ru), molybdenum (Mo), niobium (Nb), zirconium (Zr), yttrium (Y), lanthanum (La), platinum (Pt), silicon (Si), cerium(Ce), tellurium (Te), such as CoZnInSnOx, CuSnColnOx, CoZnCrNiOx, SnTbCoOx, SnTbZnOx, CoTbInOx, CoNiTbOx, CoCeNiCuOx, ZnSnTeOx, CoZnlnOx, CuSnlnOx, CoCrNiOx, SnWLaOx, SnInLaCoOx, CoOx, CoTbOx with different compositions of each chemical element, x=0.01-1.

The sensor array (e.g., sensor array 205) comprises at least one or two or three or four or eight or twelve or more gas sensors, which may be used for detecting one or more gases from metabolite gas mixtures emanated from pathogens of a wound infection.

In some embodiments, the sensor further comprises a substrate and a plurality of electrodes on the substrate.

In some embodiments, the sensor is configured in a form selected from the group consisting of a capacitive sensor, a resistive sensor, a chemiresistive sensor, an impedance sensor, and a field effect transistor sensor. Each possibility represents a separate embodiment of the present invention. In example embodiments, the sensor is configured as a chemiresistive sensor.

In certain embodiments, the sensor further comprises a detection mechanism comprising a device for measuring changes in resistance, conductance, alternating current (AC), frequency, capacitance, impedance, inductance, mobility, electrical potential, optical property or voltage threshold. Each possibility represents a separate embodiment of the present invention.

In particular embodiments, a sensor array is provided for diagnosing wound infection caused by pathogens in a subject, the sensor array having a plurality of sensors, for example between 2 and 6 and 8 and 12 and 32 and 48 sensors consisting essentially of at least two of sensing materials, a substrate, a plurality of electrodes on said substrate, and a detection mechanism.

In certain embodiments, upon contact with at least one VOC indicative of wound infection caused by pathogens such as Staphylococcus epidermidis, Streptococcus pyogenes, Enterococcus faecium, Staphylococcus aureus, Klebsiella pneumonia, Acinetobacter baumannii, Pseudomons aeruginosa, Enterobacter species, Escherichia coli, Proteus mirabilis, Serratia marcescens, Enterobacter clocae, Acetinobacter anitratus, Lactobacillus Delbrueckii, Gardnerella vaginalis, and antibiotic-resistant strains on the sensing materials, the electrical conductivity between the electrodes changes thereby providing a measurable signal indicative of wound infection.

The gas sensors may comprise sensing materials. In some examples, the sensing materials comprise at least one or two or more or mixture nanoporous structure, like mesoporous, macroporous, microporous, nanoporous, non-porous, hierarchical porous materials, including mesoporous/macroporous hierarchical structure, microporous/macroporous hierarchical structure, microporous/mesoporous hierarchical structure, microporous/mesoporous/macroporous hierarchical structure, etc. The mesoporous structure is in a configuration selected from a well-ordered mesoporous structure with regular pore arrangement, a worm-like mesoporous structure with uniform pore size but without long-range regularity, or a non-order mesostructure with pore size from 2-50 nm. The macroporous structure is a configuration selected from a well-ordered macroporous structure or non-order macroporous structure with pore size from 50 nm to 50 μm. In a further embodiment, the pore size of sensing materials is ranging from 0.4 to 2 nm, 2-50 nm, 50 nm to 200 nm, 200 nm to 500 nm, 500 nm to 1 μm, 1-50 μm. The specific surface area is 1-1000 m²/g.

The sensing material of some embodiments of the present invention may be in the form of nanomaterials. Examples of nanomaterials include nanowire, nanorod, nanosphere, nanoporous, nanoplate, nanosheet, nanomesh, nanotube, nanocube, nano-hollow sphere, nanopolyhedron, and nanoscroll, etc.

In some embodiments, the sensing material comprises unary, binary, ternary, quaternary, quinary, senary, septenary, and octonary multiple-component metal oxides, selected from an element group of tin (Sn), terbium (Tb), cobalt (Co), zinc (Zn), indium (In), copper (Cu), nickel (Ni), chromium (Cr), manganese (Mn), tungsten (W), titanium (Ti), vanadium (V), iron (Fe), aluminum (Al), gallium (Ga), silver (Ag), gold (Au), palladium (Pd), rhodium (Rh), ruthenium (Ru), molybdenum (Mo), niobium (Nb), zirconium (Zr), yttrium (Y), lanthanum (La), platinum (Pt), silicon (Si), cerium(Ce), tellurium (Te), such as CoZnInSnOx, CuSnColnOx, CoZnCrNiOx, SnTbCoOx, SnTbZnOx, CoTbInOx, CoNiTbOx, CoCeNiCuOx, ZnSnTeOx, CoZnInOx, CuSnInOx, CoCrNiOx, SnWLaOx, SnInLaCoOx, CoOx, CoTbOx with different compositions of each chemical element, x=0.01-1.

In some embodiments, the sensing material comprises unary, binary, ternary, quaternary, quinary, and senary single or multiple-component elementary metal nanoparticles, selected from an element group of silver (Ag), gold (Au), palladium (Pd), rhodium (Rh), ruthenium (Ru), platinum (Pt), osmium (Os), iridium (Ir). The size of the nanoparticles is ranging from 0.5 nm to 500 nm. The shape of the nanoparticles can be nanosphere, nanorods, nanowire, nanodot, nanostar, nanosheet, nanopalte, nanotube, nano-hollow sphere, nanocube, nanopolyhedron. Some examples are listed as follows: Pt nanospheres, Au nanodots, Ag nanowires, Ag—Au nanocubes, Os nanorods, etc.

In some embodiments, the sensing material comprises carbon-based material. The carbon-based material may be carbon blacks, active carbon, microporous carbon, mesoporous carbon, micro-mesoporous carbon, pyrolytic carbon, carbon nanotube, carbon nanofiber, carbon nanospheres, carbon nanosheet, carbon nanowire, carbon nanorod, graphene, graphene oxide and reduced graphene oxide. The carbon based material may be doped with sulfur, nitrogen, oxygen, boron, fluorine, phosphorus, selenium, chlorine, etc.

In some embodiments, the sensing material comprises carbon material that is functionalized with organic agents, including amines, fatty acids, alcohols, thiols, aldehydes, phenols, esters, epoxy, polymers, silane coupling agents, and their mixture.

In some embodiments, the sensing material comprises carbon material with single-atom metal in its carbon framework. The single-atom metal is selected from an element group of tin (Sn), terbium (Tb), cobalt (Co), zinc (Zn), indium (In), copper (Cu), nickel (Ni), chromium (Cr), manganese (Mn), tungsten (W), titanium (Ti), vanadium (V), iron (Fe), aluminum (Al), gallium (Ga), silver (Ag), gold (Au), palladium (Pd), rhodium (Rh), ruthenium (Ru), molybdenum (Mo), niobium (Nb), zirconium (Zr), yttrium (Y), lanthanum (La), platinum (Pt), silicon (Si), cerium(Ce), tellurium (Te), osmium (Os), iridium (IR).

In some embodiments, the sensing material comprises both metal and metal oxide as components to form composite sensing materials, with metal nanoparticles and metal oxides.

In some embodiments, the sensing material comprises carbon-based material as components to form composite sensing materials with metal nanoparticles and metal oxides,.

In some embodiments, the sensing material comprises conjugated polymers, such as polythiophene materials, polyaniline materials, polypyrrole materials, polycarbazole materials, etc. In some embodiments the conjugated polymers is mixed with carbon-based materials.

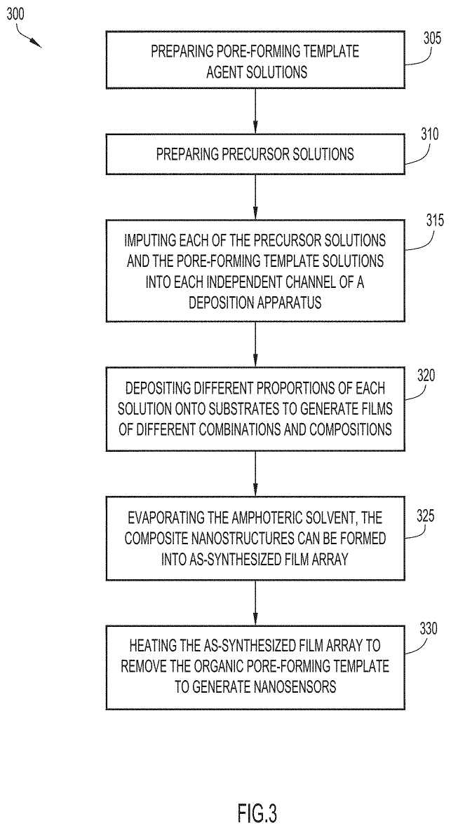

FIG. 3 shows a flow chart of an example method 300 for preparing sensing materials of sensors in a sensor array (e.g., forming a three-dimensional macroporous/mesoporous material array). In step 305, the pore-forming template agent solutions are prepared by using one kind or a mixture of two or more kinds of polymer nanospheres, carbon blacks, carbon nanotubes, carbon nanofibers, carbon nanospheres, and poly (methyl methacrylate (PMMA) microspheres, polystyrene nanosphere, latex spheres, and inorganic nanoparticles, silica nanoparticles, carbon nanoparticles, carbon dots, carbon nanocells, polymer with amphoteric solvent. In step 310, the precursor solutions are prepared to generate a target product by using one kind or a mixture of two or more kinds of metal species, graphene oxide, MXene, carbon nanotubes, metal nanoparticles, oligomeric organosilicate (1.4-bis (triethoxysilyl) benzene, 1.2-Bis(triethoxysilyl)ethane, Bis[(3-trimethoxysilyl) propylamine (amine), Ethyltriethoxysilane) with amphoteric solvent. In step 315, each of the precursor solutions and the pore-forming template agent solutions are inputted into an individual one of channels of a deposition apparatus as an independent solution. In step 320, different proportions of each independent solution are deposited onto a substrate to generate different combinations and compositions of as-synthesized film on a spot of an array. Each dot has independent compositions of at least one of the precursor solutions and at least one of the pore-forming template agent solutions. In step 325, the amphoteric solvent is evaporated, and the composite meso/macrostructures can be formed into an as-synthesized film array. In step 330, the as-synthesized film array is heated to remove the organic pore-forming template, and/or the film array is treated by NaOH or HF aqueous solution to remove silica template, to generate a 3D macroporous and mesoporous structured material array.

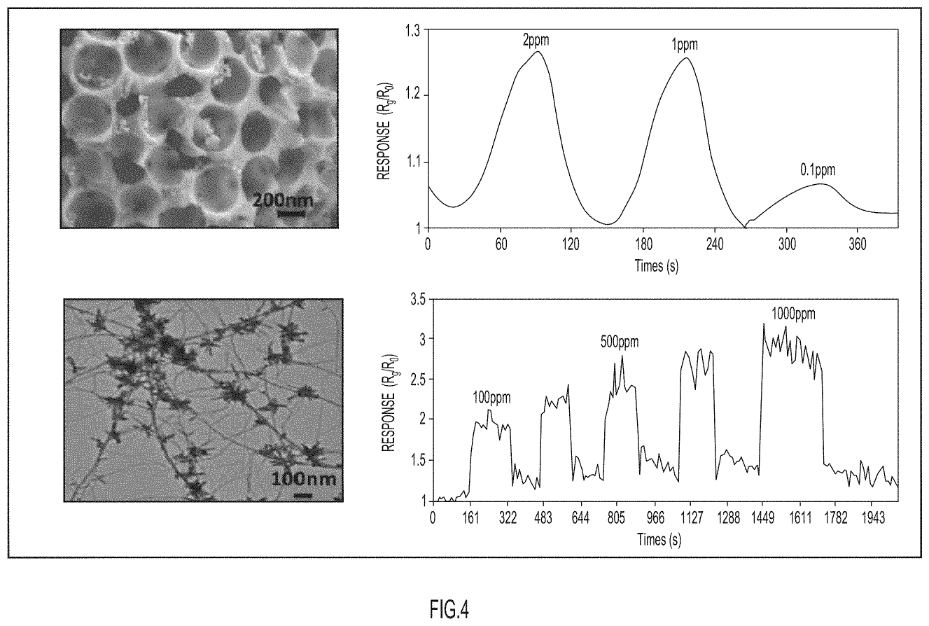

FIG. 4 shows example TEM images and response of VOC gases of sensing materials in a sensor array.

An embodiment of the present invention detects and identifies the wound infection caused by pathogens in a subject. A method (e.g., performed by the devices described herein) comprises providing a sensor array comprising a plurality of sensors, for example between 2 and 6 and 8 and 12 and 32 sensors, the sensors comprising at least one or two or more or mixture nanoporous structure, like mesoporous, macroporous, microporous, nanoporous, non-porous, hierarchical porous materials, including mesoporous/macroporous hierarchical structure, microporous/macroporous hierarchical structure, microporous/mesoporous hierarchical structure, microporous/mesoporous/macroporous hierarchical structure, etc. The mesoporous structure is in a configuration selected from a well-ordered mesoporous structure with regular pore arrangement, a worm-like mesoporous structure with uniform pore size but without long-range regularity, or a non-order mesostructure with pore size from 2-50 nm. The macroporous structure is a configuration selected from a well-ordered macroporous structure or non-order macroporous structure with pore size from 50 nm to 50 μm. In a further embodiment, the pore size of sensing materials is ranging from 0.4 to 2 nm, 2-50 nm, 50 nm to 200 nm, 200 nm to 500 nm, 500 nm to 1 μm, 1-50 μm. The specific surface area is 1-1000 m²/g.

The sensing material comprises unary, binary, ternary, quaternary, quinary, senary, septenary, and octonary multiple-component metal oxides, selected from an element group of tin (Sn), terbium (Tb), cobalt (Co), zinc (Zn), indium (In), copper (Cu), nickel (Ni), chromium (Cr), manganese (Mn), tungsten (W), titanium (Ti), vanadium (V), iron (Fe), aluminum (Al), gallium (Ga), silver (Ag), gold (Au), palladium (Pd), rhodium (Rh), ruthenium (Ru), molybdenum (Mo), niobium (Nb), zirconium (Zr), yttrium (Y), lanthanum (La), platinum (Pt), silicon (Si), cerium(Ce), tellurium (Te), such as CoZnInSnOx, CuSnColnOx, CoZnCrNiOx, SnTbCoOx, SnTbZnOx, CoTbInOx, CoNiTbOx, CoCeNiCuOx, ZnSnTeOx, CoZnInOx, CuSnInOx, CoCrNiOx, SnWLaOx, SnInLaCoOx, CoOx, CoTbOx with different compositions of each chemical element, x=0.01-1.

The sensor array is exposed to the metabolite gas mixtures emanated from pathogens of a wound infection in a subject. The response parameters from the sensors in a sensor array are measured and analyzed upon exposure to the test VOCs using a detection mechanism to generate response patterns. The response pattern is analyzed with a reference signal obtained from a control sample. Pathogens are recognized and identified using pattern recognition and machine learning algorithms. The infection may be diagnosed and/or bacteria may be identified in one of internal medicine, rheumatology, physical medicine, rehabilitation, clinical research, and basic research in the fields of immunology and/or microbiology. The efficacy of a drug to a subject may be evaluated where the drug is known to kill or inhibit the growth of the bacteria causing the infection.

In some embodiments, measuring a plurality of response parameters comprises measuring, extracting, filtering, magnifying, and processing a plurality of electrical signals from the sensors.

In some embodiments, the response induced parameter is selected from the group consisting of the normalized change of sensor signal at the peak of the exposure, the normalized change of sensor signal at the middle of the exposure, the normalized change of sensor signal at the end of the exposure, and the area under the curve of the sensor signal.

Pattern recognition and machine learning algorithms are applied to train on the data and generate the correlations between certain infection issues with patterns of signals. The algorithms comprise at least one algorithm selected from the group consisting of artificial neural network algorithms, such as Naïve Bayes, principal component analysis (PCA), support vector machine (SVM), multi-layer perception (MLP), generalized regression neural network (GRNN), fuzzy inference systems (FIS), self-organizing map (SOM), radial bias function (RBF), genetic algorithms (GAS), neuro-fuzzy systems (NFS), adaptive resonance theory (ART), partial least squares (PLS), multiple linear regression (MLR), principal component regression (PCR), discriminant function analysis (DFA), linear discriminant analysis (LDA), cluster analysis, and nearest neighbor. In one embodiment, the at least one algorithm is principal component analysis (PCA).

Once the model is set up, with the VOC pattern (and optionally vital signs and/or environment conditions) as the input, the system can generate the early diagnosis result for wound infection purposes.

The pathogens of wound infection comprise one or more bacterial or fungi species, but not limited from Acetinobacter anitratus, Acinetobacter baumannii, Actinomyces israelii, Agrobacterium radiobacter, Agrobacterium tumefaciens, Anaplasma phagocytophilum, Azorhizobium caulinodans, Azotobacter vinelandii, Bacillus anthracia, Bacillus brevis, Bacillus cereus, Bacillus fusiformis, Bacillus licheniformis, Bacillus megaterium, Bacillus mycoides, Bacillus stearothermophilus, Bacillus subtilis, Bacillus thuringiensis, Bacteroides fragilis, Bacteroides gingivalis, Bacteroides melaninogenicus, Bartonella henselae, Bartonella quintana, Bordetella bronchiseptica, Bordetella pertussis, Borrelia burgdorferi, Brucella abortus, Brucella melitensis, Brucella suis, Burkholderia mallei, Burkholderia pseudomallei, Burkholderia cepacia, Calymmatobacterium granulomatis, Campylobacter coli, Campylobacter fetus, Campylobacter jejuni, Campylobacter pylori, Chlamydia trachomatis, Chlamydophila pneumoniae, Chlamydophila psittaci, Clostridium botulinum, Clostridium difficile, Clostridium perfringens, Clostridium tetani, Corynebacterium diphtheriae, Corynebacterium fusiforme, Coxiella burnetii, Ehrlichia chaffeensis, Enterobacter clocae, Enterococcus avium, Enterococcus durans, Enterococcus faecalis, Enterococcus faecium, Enterococcus galllinamm, Enterococcus maloratus, Escherichia coli, Francisella tularensis, Fusobacterium nucleatum, Enterobacter species, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumonia, Lactobacillus acidophilus, Lactobacillus bulgaricus, Lactobacillus casei, Lactobacillus Delbrueckii, Lactococcus lactis, Legionella pneumophila, Listeria monocytogenes, Methanobacterium extroquens, Microbacterium multiforme, Micrococcus luteus, Moraxella catarrhalis, Morganella morganii, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Mycoplasma mexican, Neisseria gonorrhoeae, Neisseria meningitidis, Pasteurella multocida, Pasteurella tularensis, Porphyromonas gingivalis, Prevotella melaninogenica, Proteus vulgaris, Proteus mirabilis, Proteus penneri, Providencia stuartii, Pseudomons aeruginosa, Pseudomonas aeruginosa, Rhizobium radiobacter, Rickettsia prowazekii, Rickettsia psittaci, Rickettsia quintana, Rickettsia rickettsii, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Spirillum volutans, Staphylococcus aureus, Staphylococcus epidermidis, Stenotrophomonas maltophilia, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema pallidum, Treponema denticola, Vibrio cholerae, Vibrio comma, Vibrio parahaemolyticus, Vibrio vulnificus, Yersinia enterocolitica, Yersinia pestis and Yersinia pseudotuberculosis, and/or known to comprise one or more antibiotic-resistant strains descending from a known species, and/or known to comprise one or more extended spectrum beta-lactamase-producing strains descending from a known species, in particular the one or more extended spectrum beta-lactamase-producing strain is selected from the group consisting of: extended spectrum beta-lactamase-producing Escherichia coli, and extended spectrum beta-lactamase-producing Klebsiella pneumoniae.

Antibiotic-resistant bacterial strains are selected from the group consisting of: Carbapanem-resistant Acinetobacter baumannii, carbapanem-resistant Pseudomonas aeruginosa, vancomycin-resistant Enterococcus faecium, methicillin-resistant Staphylococcus aureus, vancomycin-resistant Staphylococcus aureus, clarithromycin-resistant Helicobacter pylori, fluoroquinolone-resistant Campylobacter coli, fluoroquinolone-resistant Campylobacter fetus, fluoroquinolone-resistant Campylobacter jejuni, fluoroquinolone-resistant Campylobacter pylori, fluoroquinolone-resistant Salmonella enteritidis, fluoroquinolone-resistant Salmonella typhi, fluoroquinolone-resistant Salmonella typhimurium, cephalosporin-resistant Neisseria gonorrhoeae, fluoroquinolone-resistant Neisseria gonorrhoeae, penicillin-non-susceptible Streptococcus pneumonia, ampicillin-resistant Haemophilus influenza, fluoroquinolone-resistant Shigella dysenteriae, carbapanem-resistant Escherichia coli, carbapanem-resistant Klebsiella pneumonia, carbapanem-resistant Enterobacter cloacae, carbapanem-resistant Serratia marcescens, carbapanem-resistant Proteus vulgaris, carbapanem-resistant Proteus mirabilis, carbapanem-resistant Proteus penneri, carbapanem-resistant Providencia stuartii, carbapanem-resistant Morganella morganii, cephalosporin-resistant Escherichia coli, cephalosporin-resistant Klebsiella pneumonia, cephalosporin-resistant Enterobacter cloacae, cephalosporin-resistant Serratia marcescens, cephalosporin-resistant Proteus vulgaris, cephalosporin-resistant Proteus mirabilis, cephalosporin-resistant Proteus penneri, cephalosporin-resistant Providencia stuartii and cephalosporin-resistant Morganella morganii.

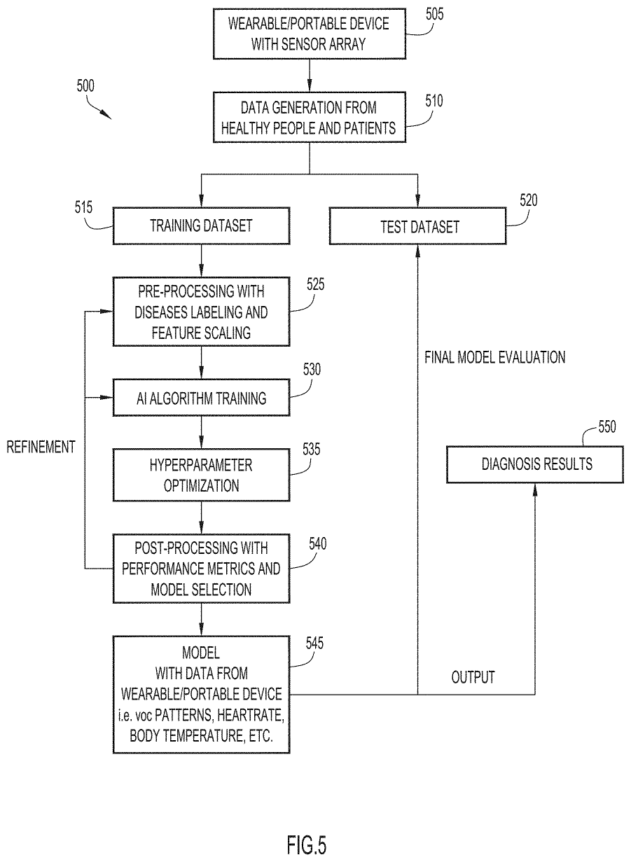

FIG. 5 is a flow chart showing a method 500 diagnosing wound infection using the VOC data (e.g., for a wearable/portable device 505). First, a sufficient number of data samples from both healthy people (clean wound) and patients with known health conditions (wound infections), i.e., a discovery cohort, are collected at step 510. The data samples include VOC data patterns or VOC data patterns and vital signs and/or environment conditions. Mathematical algorithms are used to train on the data, identify the distinct pattern between healthy controls and patients, and generalize a classifier at steps 525, 530, 535, and 540. The mathematical algorithm can be one or more of PCA, Naive Bayes, support vector machine (SVM), multi-layer perception (MLP), generalized regression neural network (GRNN), fuzzy inference systems (FIS), self-organizing map (SOM), radial bias function (RBF), genetic algorithms (GAS), neuro-fuzzy systems (NFS), adaptive resonance theory (ART), partial least squares (PLS), multiple linear regression (MLR), principal component regression (PCR), discriminant function analysis (DFA), linear discriminant analysis (LDA), cluster analysis, and nearest neighbor. The classifier is preferably a machine learning model, but may alternatively be a mathematical equation of a partial of vital signs and/or skin (or pathogen)-VOCs to predict wound infection.

In the discovery cohort, a portion of the data is randomly assigned into a training set at step 515, while the remainder is assigned to the test set at step 520. The optimal classifiers are developed in the training set using the test set. The values of the area under the ROC curve (AUC) in patients are determined. Then, the sensitivity, specificity, positive predictive value (PPV), negative predictive value (NPV), and accuracy of the device for both training and test groups are evaluated. For example, a 5-fold cross-validation (randomly select one-fold of samples for the testing, the remaining 4 folds for training) can be applied to calculate the classification performance of the training set.

Once the mathematical model (aka classifier) is developed at step 545, one and more independent clinical cohorts are collected to validate the model. In the process, the model parameters are refined (at steps 525, 530, 535, 540) and the patients may further be stratified into subtypes that use different sets of parameters. After model validation and refinement, users can input into the model/classifier VOC data or vital signs from a subject or both (and optionally environment conditions) and the model may predict a health condition (or wound infection) at step 550.

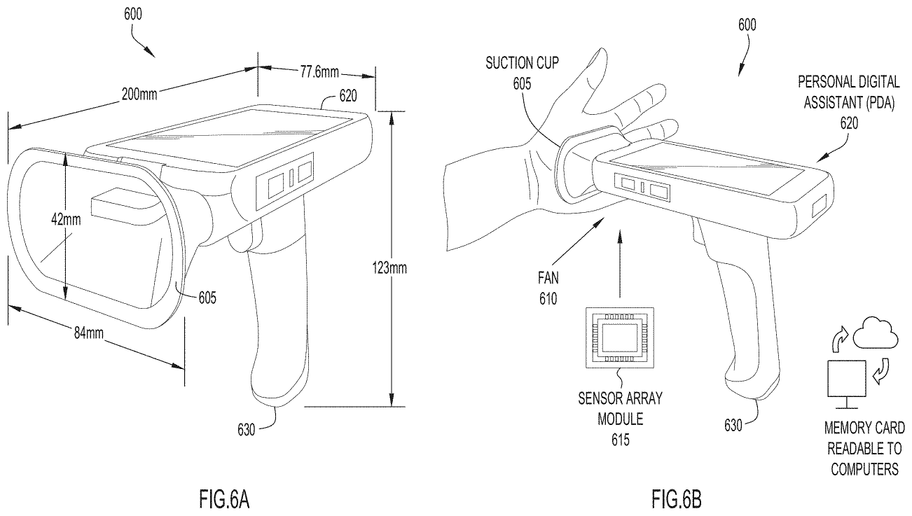

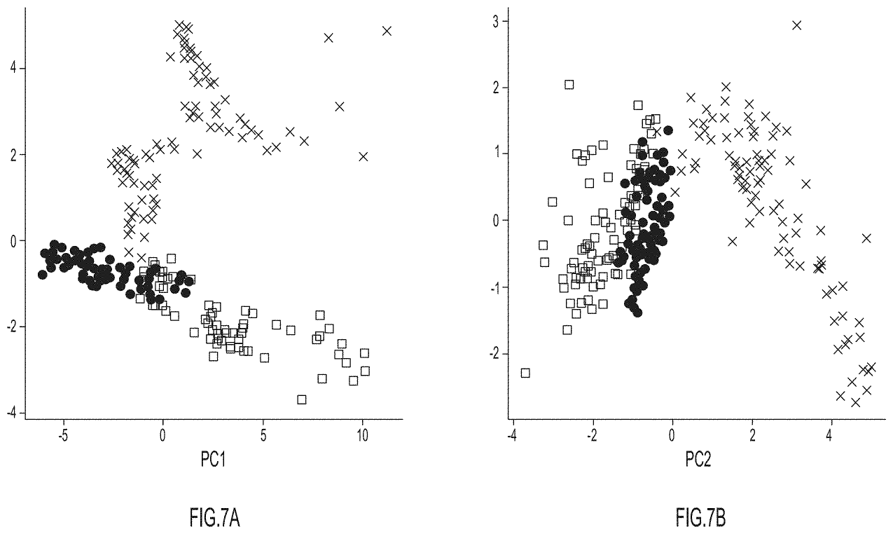

FIGS. 6A and 6B show an example portable device and how it may be used according to an embodiment of the present invention. Portable device 600 includes a handle 630 coupled to an underside of a processing device (e.g., Personal Digital Assistant (PDA) 620). The portable device has a disposable suction cup 605 adapted to cover a surface of a subject, a fan 610 adapted to create a slight vacuum in the suction cup 605, at least one sensor array module 615, and PDA 620 with an interface that a user may interact with. The sensor array module 615 is similar to the one described above (e.g., sensor array 205). It contains at least one sensor array, at least one sensor signal processing circuit, at least one switch channel circuit, at least one analog-to-digital converter (ADC), at least one Micro-controller Unit (MCU), at least one power management system, and at least one USB interface. The signal processing circuit may include a voltage divider circuit to measure resistance for each sensor of the sensor array. To ensure ADC accuracy, multiple ADCs and the switch channel are used for data acquisition and digitalization. The MCU collects digital signals from ADCs and transmits it to the System-On-Chip (SOC) on the PDA 620.

The sensor array module 615 is attached to the suction cup 605. Employing the suction cup 605 reduces interference from the environment, e.g., hand sweat, dirt, temperature changes. To increase the sensitivity of the device, a fan 610 is used to create a slight negative pressure in the suction cup so that VOCs emanated from the palm mostly enter the portable device. The PDA 620 contains SOC with 1-1/wireless/USB communication capability, central processing unit (CPU), memory, and an OLED or LCD screen. The data can be transferred by USB cable or wireless communication to a terminal (e.g., a PC) or cloud database. The PDA runs an APP to provide the human-computer interface, test data collection, and data transfer for further analysis. The test results may be shown on the PDA as a number or, more visibly, using color coded messages (e.g., white with data of 0 may mean Blank; green with data of 0.1-2.9 may mean Health (no infection), yellow with data of 3-4.9 may mean Continue Testing, and red with data over 5.0 may mean Alert). For example, when data is over 5.0, the PDA alerts the user by showing a red message.

Variations of the device 600 are multiple. For example, the device may not have a fan to pull vacuum and rely on diffusion. Further, the device may be handheld or stationary. In some embodiments, the device has a pressure sensor that can detect a change in the ambient pressure and turn on the device from a standby mode to a work mode. As such, when a subject's body part (e.g., hand, forehead) covers the suction cup 605, the change in pressure may turn the device to a work mode. In an embodiment, the device may turn off automatically when the data is sufficient for readout or after a predetermined period of time in the standby mode. The device may have a manual entry option through which a user can manually set a time for test, e.g., for 0.001-30 minutes. In addition, a health management system may be installed on a remote device (e.g., server or smartphone) to analyze and visualize sensor readouts.

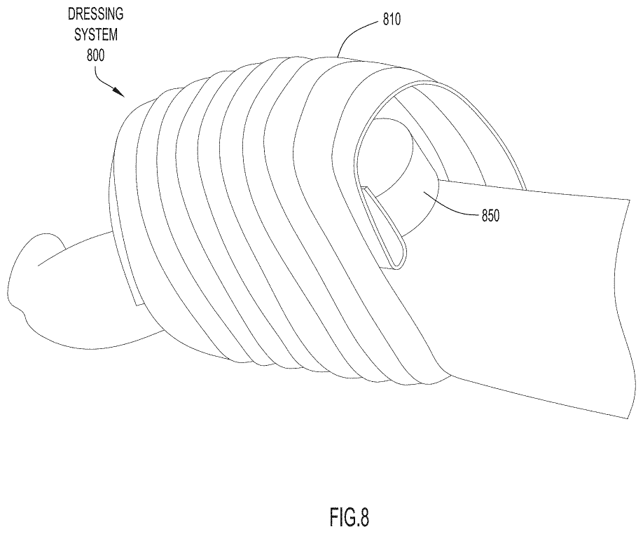

FIGS. 7A and 7B show analysis of VOCs patterns detected using the portable device 600 for monitoring the growth of three bacteria (Escherichia coli (E. coli), Pseudomonas aeruginosa (PA), and Staphylococcus aureus (SA)) in a wound infection. Each dot represents the device readout of a VOC in a principal space.

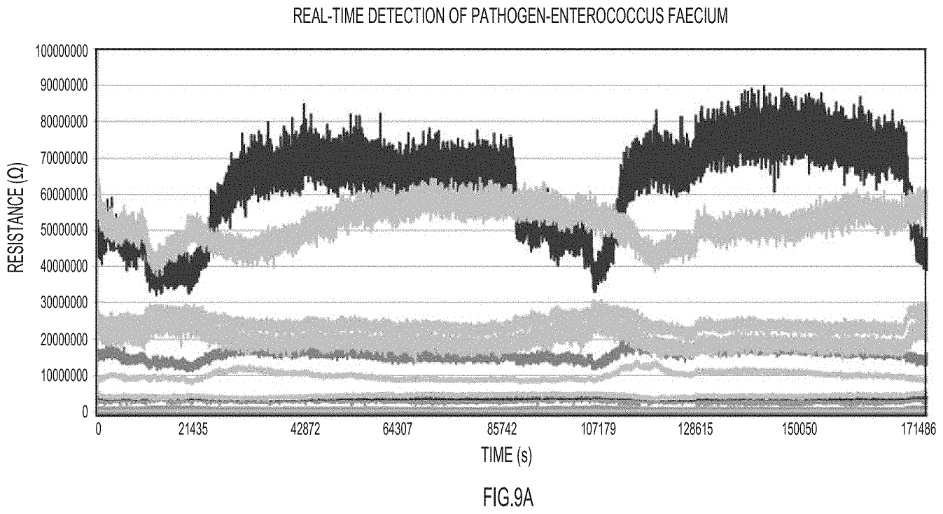

FIG. 8 illustrates an example embodiment of a wearable device 850 for real-time monitoring of a wound infection. Wearable device 850 is integrated into a dressing system 800 for detection, identification and monitoring of bacteria that cause wound infection. The dressing system 800 includes wound dressing 810 that covers the wound, and wearable device 850 that is coupled to the wound dressing and placed over or in vicinity of the wound.

Similar to what has been described with respect to FIG. 1 or 2, the wearable device 850 has a sensor array, a sensor signal processing circuit (voltage divider circuit), a 4-channel switching circuit, four 14-bit analog-to-digital converters (ADCs), a Micro-controller Unit (MCU), and a USB interface. The sensor array may contain a 3×3 array with 9 different gas sensors and one physiological sensor (skin temperature). Each of the 9 sensors has different nano structured multiple-component metal oxides or different amount (e.g., concentration) of nano structured multiple-component metal oxides make up or composition. The voltage divider circuit is used to process changes in properties (e.g., voltage changes, resistance changes, impedance changes, combinations of these and the like) in each sensor in the sensor array. To ensure the ADC accuracy, 4 ADCs and a 4-channel switching circuit is used for data acquisition and digitalization.

The MCU collects digital signals from ADCs and transmits it to the system. The data is transferred wirelessly to a PC and cloud database. A controller unit comprises an 8-bit microcontroller with a Wi-Fi communication module, a Liquid Crystal Display (LCD), a Capacitive Touch Panel (CTP), and an “on-off switch”. A communication unit sends and receives radio waves at a certain frequency. A communication and power supply module (rechargeable battery) contains the power source and is also responsible for data acquisition and transmission, and can be connected with a PC through the Wi-Fi communication module. CTP and LCD provide the capability of human interaction interface.

The wearable device 850 continuously detects VOCs emanated from ESKAPEE pathogens (Enterococcus faecium, Staphylococcus aureus, Klebsiella pneumoniae, Acinetobacter baumannii, Pseudomonas aeruginosa, Enterobacter species, and Escherichia coli) for 36 hours. Sixteen sensors had complete profiles for all the experiments.

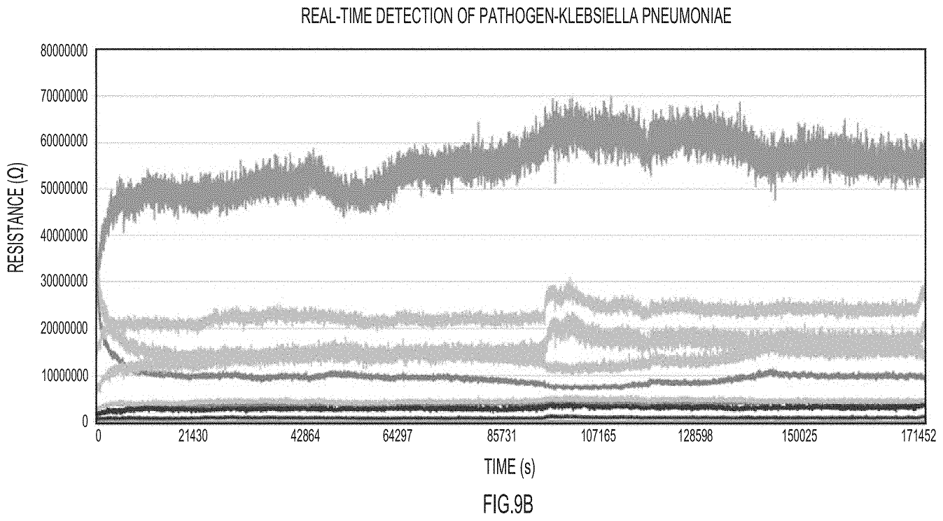

FIGS. 9A-9D present the measurement results by the sixteen sensors of four analytes (Enterococcus faecium, Klebsiella pneumoniae, Acinetobacter baumannii, Enterobacter species) within 36 hours. Each ESKAPEE pathogen can be discriminated by unique patterns within the first 12 hours. To advance AI-powered detection, a 9-class Support Vector Machine (SVM) classifier was constructed to discern the sensor profiles of bacterial strains. To avoid artifacts of overfitting, the data set from 75% of the samples, sampled every 0.5 hour from the sensor profiles, was used as a training set to train the SVM model. The remaining 25% of the sample data was used as the test set to validate the SVM model. The average accuracy was very high for each bacterium, and the overall accuracy reached 97.08.

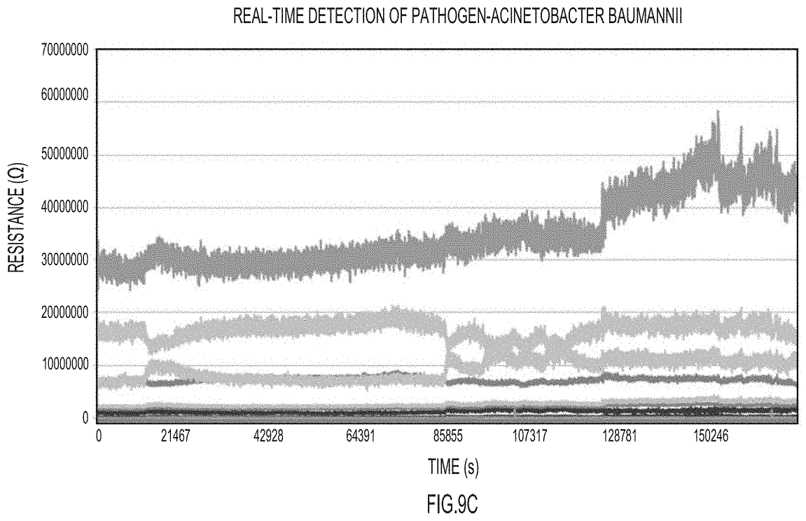

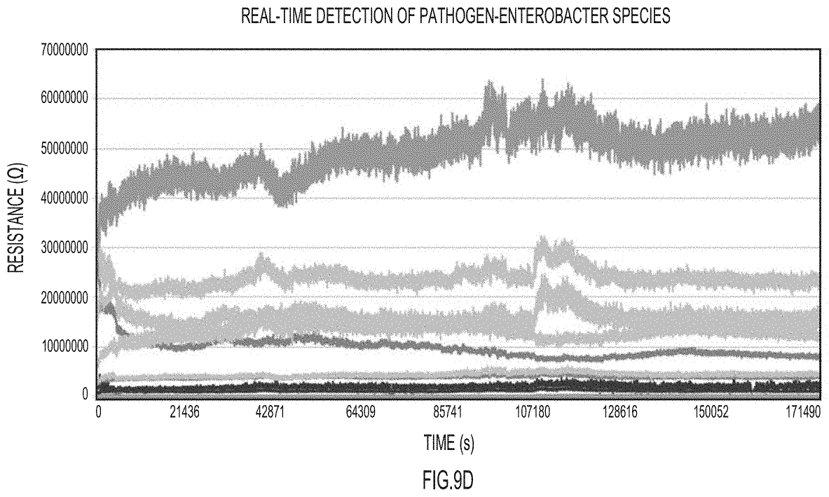

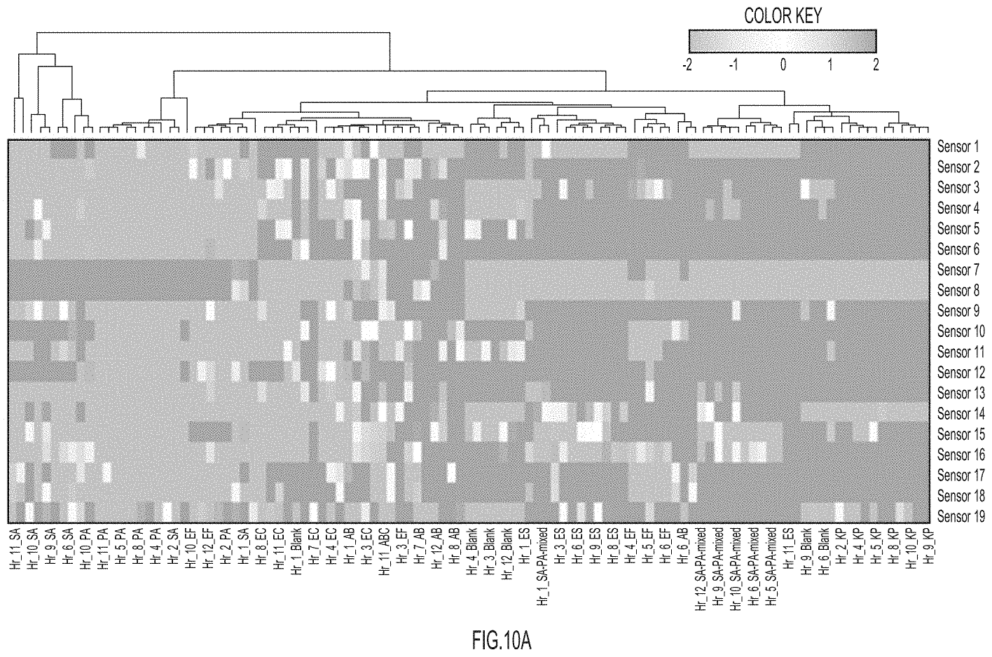

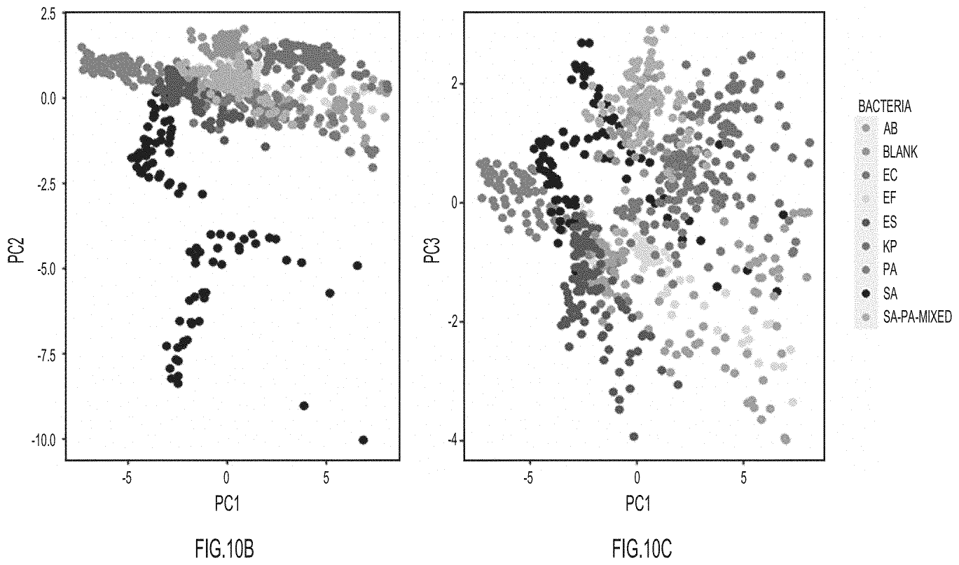

Another example embodiment shows detection and identification of seven bacteria in a wound infection. FIGS. 10A-10D show differentiation of common wound infection pathogens including Enterococcus faecium (EF), Staphylococcus aureus (SA), Klebsiella pneumonia (KP), Acinetobacter baumannii (AB), Pseudomons aeruginosa (PA), Enterobacter species (ES), and Escherichia coli (EC) (ESKAPEE) using device and PCA analysis. Each point represents a sensor array readout of bacteria in a principal space. Each of ESKAPEE bacteria was cultured on individual LB-agar plates for 24 hrs. prior to a qualitative analysis of the early emissions of VOCs from the organisms. Culture media on Luria broth (LB) agar plates without bacteria were used as a negative control for background signals. Cultures were continuously measured by the MACchip array for 48 hours. The measurement of 5:5 mixed PA-SA strains and an empty plate were also included. Applying AI algorithms, signals from individual strains were separated into clearly distinguishable clusters.

The device of this example embodiment uses a nanocomposite MACchip sensor array of 32 sensors. The MACchip sensor array integrates three types of sensors manufactured by a high-throughput 3D nano-printing process, using 200 different customized inks. The three types of sensors (and inks) use macroporous, mesoporous, microporous macro/mesoporous nanoCrystallines (MAC) film embedded with metal oxides, a MAC embedded with graphene and nanoparticles, and a carbon black and conductive polymer composite. Sensor materials enable different and complementary sensing capabilities. Sensors are designed to readily absorb chemical vapors, whereupon the electrical characteristics of sensors change after chemical binding, generating a signal. Software with AI algorithms assesses the presence/pattern of specific chemical pathogen odors, with outcomes presented to the patient and/or caregiver.

The sensing materials, such as CoZnInSnOx, CuSnCoInOx, CoZnCrNiOx, SnTbCoOx, SnTbZnOx, CoTbIn0x, CoNiTbOx, CoCeNiCuOx, ZnSnTeOx, CoZnInOx, CuSnInOx, CoCrNiOx, SnWLaOx, SnInLaCoOx, CoOx, CoTbOx with different composition of each chemical element, x=0.01-1, form a conductive path between the electrodes to generate sensors in the MACchip sensor array. For quaternary metal oxides, the molar ratio of each element is 1:1:1:1; 1:1:0.5:0.5; 0.5:0.5:1:1; 0.1:0.1:1:1; and 1:1:0,1:0.1. For ternary metal oxides, the molar ratio of each element is 1:1:1; 1:0.5:0.5; 1:0.1:0.1; 0.5:0.5:1; 0.1:0.1:1; and 0.1:1:0.1. The pore size of multiple-component is 2-100 nm.

VOCs from laboratory strains representing all ESKAPEE bacteria, a mixed culture, and an empty LB-agar plate are monitored continuously for 48 hours. 19 sensors had complete profiles for all the experiments. FIG. 10A illustrates the 19 sensor measurements of 9 analytes within 18 hours. In a hierarchical clustering heatmap, the bacterial profile tends to cluster with distinct patterns for each bacterium. The patterns were visualized using a principal component analysis (PCA), which condenses the high-dimensional data to a lower dimension, but preserved the highest variation. FIGS. 10B and 10C project the entire sensor data (19 sensors, 7 bacteria, PA-SA mixed bacteria, a blank plate, in a 0.5-hour sampling interval) into two principal components (PC1 and PC2), and (PC1 and PC3), respectively. Each ESKAPEE bacteria, mixed cultures and empty plate can be discriminated by unique patterns within the first hour, and only a couple of clusters (such as EC and EF) partially overlapped.

To advance AI-powered detection, a 9-class Support Vector Machine (SVM) classifier was constructed to discern the sensor profiles of 7 bacterial strains, PA-SA mixed strains, and an empty plate. To avoid artifacts of overfitting, the data set (sampled every 0.5 hour from the sensor profiles, totaling 804 samples for 7 bacteria, the mixed type, and blank plate) was randomly divided as a training set (75% of samples) and test set (25% of samples), the SVM model was trained in the training set, and validated using the test set. After feature selection, the 9 best features were extracted out of 32 sensors. The accuracy of prediction for each bacteria strain was recorded, and the process was repeated 10,000 times. The average accuracy was very high for each bacterium, and the overall accuracy reached 97.08% (FIG. 10D).

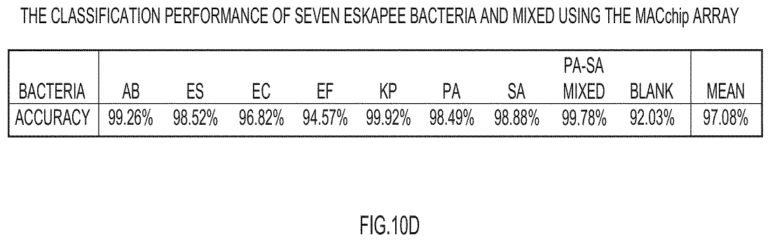

FIG. 11 illustrates a further embodiment of a wearable device 1100 for real-time monitoring. In this illustrative example, the wearable device 1100 is a wearable wristwatch. The wearable device 1100 comprises a sensor array, sensor signal processing circuit (voltage divider circuit), a 4-channel switching circuit, an analog-to-digital converter (ADCs), a Micro-controller Unit (MCU), and USB interface as part of electronics 1125 (similar to FIG. 1 or 2). By way of example, the sensor array comprises two different gas sensors and one physiological sensor (heart rate). Each of the two sensors has different nanostructured multiple-component metal oxides or different amount (e.g., concentration) of nanostructured multiple-component metal oxides make up or composition.

The voltage divider circuit is used to process an electrical change (e.g., voltage changes, resistance changes, impedance changes, combinations of these and the like) in each sensor of the sensor array. The MCU collects digital signals from ADCs and transmits it to the system. Data is analyzed using artificial intelligence algorithms and transferred by wirelessly to a PC and/or cloud database. The controller unit comprises an 8-bit microcontroller with a WiFi communication module, a Liquid Crystal Display (LCD) 1130, a Capacitive Touch Panel (CTP) 1135, and an “on-off switch”. A communication and power supply module (e.g., a rechargeable battery) 1140 contains the power source and is also responsible for data acquisition and transmission, and can be connected with a PC through the Wi-Fi communication module. The CTP and/or LCD provide the capability of human interaction interface.

In some embodiments, a negative pressure wound device may include the devices described above for wound infection detection and be used for negative pressure wound therapy.

Negative pressure wound therapy (NPWT) is a treatment modality that involves the use of a device to apply negative pressure to a wound dressing in order to promote wound healing and reduce the risk of infection. NPWT has been shown to be effective in a variety of wound types, including chronic wounds, traumatic wounds, and surgical wounds.

Despite the benefits of NPWT, wound infection remains a common complication of the therapy. Wound infection can lead to delays in healing and may require additional treatment, such as the use of antibiotics.

Accordingly, an embodiment of the present invention provides a negative pressure wound device that includes one or more integrated gas sensors for the early detection and prediction of wound infections caused by pathogens (such as bacteria and fungi).

The negative pressure wound device is designed to continuously monitor the wound healing and provide real-time data on the presence and levels of gases indicative of infection. This allows for timely interventions (e.g., clean wound, change dressing, drugs, etc.) to prevent or treat the infection, improving the overall treatment of the wound.

An embodiment of the present invention relates to a negative pressure wound device incorporating gas sensors for the continuous monitoring of wound healing and early detection of wound infections caused by pathogens (such as bacteria and fungi). The negative pressure wound device includes a flexible wound dressing connected to a vacuum pump and a gas sensor system or module. The gas sensor module is configured to detect the presence of certain gases, such as hydrogen, oxygen, methane, carbon dioxide, ammonia, and volatile organic compounds, which may indicate the presence of an infection in the wound. The gas sensor module is also configured to detect the presence of specific gases produced by bacteria and fungi, allowing for the early detection and prediction of specific types of infections. The gas sensor module is connected to a control unit, which analyzes the gas levels and alerts the caregiver if an infection is detected or predicted. The negative pressure wound device may also include a display for displaying the results of the gas sensor readings, and may be configured to transmit the sensor readings to a remote device for analysis and/or storage. The device can be connected to a wireless communication system for transmitting data to a remote monitoring system or healthcare provider. The integration of the gas sensor module into the negative pressure wound device provides real-time wound monitoring and allows for early detection and prediction of wound infections, which can improve patient care and outcomes and may help to reduce the use of antibiotics.

In an embodiment, the negative pressure wound device includes a housing that encloses the components of the device, including a negative pressure source and the gas sensors. The negative pressure source applies a negative pressure to the wound site, which helps to remove excess fluid and promote healing. The gas sensors are configured to measure the levels of specific gases produced by bacteria and fungi present in the wound. The data from the gas sensors is transmitted to a control unit, which is configured to receive and analyze the data. The control unit is also responsible for controlling the negative pressure source in response to the data from the gas sensors. For example, the negative pressure source may be enabled in response to detection of an infection, pumping rate adjusted in response to progression or remission of infection, etc.

The device includes a flexible wound dressing connected to a vacuum pump and a gas sensor module. The gas sensors are positioned within the housing of the device, and can be of various types, including electrochemical, metal oxides, infrared, or optical sensors.

The gas sensors are configured to detect gases present within the wound dressing, such as hydrogen, oxygen, methane, carbon dioxide, ammonia, and volatile organic compounds (VOCs), which may indicate the presence of an infection in the wound. VOCs include, but are not limited to, aldehydes, alcohols, ketones, acids, Sulphur containing compounds, esters, hydrocarbons and nitrogen containing compounds, propene, acetaldehyde, ethanol, acetonitrile, (E)-2-Butene, (Z)-2-butene, 2-propenal, n-propanol, Acetone, 2-propanol, dimethyl sulfide, 1-pentene, isoprene, n-Pentane, 1,3-Dioxolane, 2-methyl-2-propenal, 2-methyl-Propanal, 3-Buten-2-one, 2-methyl Furan, n-Butanal, 2-Butanone, 3-methyl Furan, Ethyl Acetate, 2-Butenal, 2-methyl-1,3-Dioxolane, 2-methyl-2-Pentene, 2,3-dimethyl-2-Butene, (E)-2-Methyl-1,3-pentadiene, (Z)-2-Methyl-1,3-pentadiene, 3-methyl-Butanal, 2-methyl-Butanal, Isopropyl acetate, 2-Pentanone, 2,5-dimethyl Furan, allyl methyl Sulfide, n-Pentanal, 3-methyl-2-Butenal, 1-Heptene, 2-Heptene, n-Heptane, 2-ethyl-Butanal, 4-Methyl-3-penten-2-one, Isobutyl acetate, 2-Hexanone, n-Hexanal, gamma-Butyrolactone, n-Butyl acetate, (E)-2-Hexenal, 1-Octene, n-Octane, 2-Heptanone, n-Heptanal, Benzaldehyde, 1-Nonene, n-Nonane, 6-Methyl-5-hepten-2-one, 2-pentyl-Furan, b-Pinene, n-Octanal, p-Cymene, DL-Limonene, Styrene, Eucalyptol, n-Nonanal, 2-Ethylhexanol, 3-Methylhexane, Butyraldehyde, Ethylbenzene, Ethyl butanoate, toluene, undecane, H2O, CO, NO, N2O, NO2, ammonia, Acetophenone, 4-methylphenol, Dodecane, Dimethyl pyrazine, 2-Pentanol, 2-butanol, 2-pentene, 2-methylbutyl isobutyrate, 2-methoxy-5-methylthiophene, amyl isovalerate; 2-methylbutyl 2-methylbutyrate, 6-tridecane, 3-methyl 1H-pyrrole, 2-methyl (2-propenyl)-pyrazine, 2,3-dimethyl-5-isopentylpyrazine, Methyl thiolacetate, Methyl thiocyanate, Hydrogen cyanide, 2-aminoacetophenone, 1-undecene, Formaldehyde, Dimethyl ether, carbon dioxide, pentafluoropropionamide, Methyl cyclohexane, 2-methylbutanol, N-propyl acetate, Butanal, 2,5-dimethyltetrahydrofuran, Carbon disulfide, methyl propanoate, methyl butanoate, 6-methyl-5-hepten-2-one, 2,5-dimethylpyrazine, Hydrogen sulfide, Propanol, Indole, 1,1,2,2-tetrachloroethane, Butanol, 2-tridecenone, 3-hydroxy-2-butanone, 1-hydroxy-2-propanone, 3-nitro-benzenesulfonic acid, Isobutyric acid, methyl ester, 1,2-dimethyl-benzene, 2-ethyl-1-hexanol, Isopentyl 3-methylbutanoate, 2,4-dinitro-benzenesulfonic acid, Decanal, 2-methyl-1-propanol, 2-phenylethanol, 1,4-dichlorobenzene, 2-methylbutanoic acid, methyl mercaptan, 2-nonanone, 3-methyl-1-butanol, 3-methylbutanoic acid, dimethyl trisulfide, dimethyl disulfide, and acetic acid.

In still other embodiments, one or more of the plurality of sensor arrays contains a plurality of physiological sensors, each physiological sensor is adapted to detect at least one parameter selected from heart rate, pulse rate, respiratory rate, blood oxygen saturation, blood pressure, hydration level, stress, position and balance, body strain, neurological functioning, brain activity, blood pressure, cranial pressure, auscultatory information, skin and body temperature, eye muscle movement, sleep, cholesterol, lipids, blood panel, body fat density, muscle density, temperature, humidity, and pressure.

Some embodiments of the negative pressure wound device are capable to measure skin and body temperature (−15° C. to 45° C.), heart rate, humidity (0-99%), and a variety of concentrations of VOCs. The VOC detection limit may range from 0.1 ppb to 5000 ppm, e.g., 0.1 ppb-1 ppb, 1 ppb-5 ppb, 5 ppb-10 ppb, 10 ppb-50 ppb, 50 ppb-100 ppb, 100 ppb-200 ppb, 200 ppb-300 ppb, 300 ppb-500 ppb, 500 ppb-1 ppm, 1 ppm-2 ppm, 2 ppm-5 ppm, 5 ppm-10 ppm, 10 ppm-100 ppm, 100 ppm-200 ppm, 200 ppm-500 ppm, 500 ppm-1000 ppm, 1000 ppm-2000 ppm, and 2000 ppm-5000 ppm.

The gas sensor system is also configured to detect the presence of specific gases produced by bacteria and fungi, allowing for the early detection and prediction of specific types of infections.

The negative pressure wound device may also include a display for displaying the results of the gas sensor readings, and may be configured to transmit the sensor readings to a remote device for analysis and/or storage. The remote device may be configured to provide an indication of wound infection and type of pathogens based on the analysis of the gas sensor readings.

The negative pressure wound device may also include a treatment module for taking timely interventions to prevent or treat the infection based on the data from the gas sensors.

In one embodiment, the negative pressure wound device includes a housing with a wound dressing attached to the housing. The housing may be shaped and sized to fit over the wound, and the wound dressing may be made of a porous material that allows for the passage of gases.

A pump is in communication with the housing and is configured to apply negative pressure to the wound dressing. The pump may be a mechanical or electrical pump, and may be powered by a battery or other power source.

The negative wound pressure device also includes a processor that is connected to the gas sensor system. The processor is configured to analyze the data collected by the gas sensor system and to generate an alert when the data indicates the presence of a bacterial or fungal infection at the wound site. The alert may be presented on a display that is connected to the processor. The display may be a separate display unit or may be incorporated into the negative wound pressure device.

The negative wound pressure device may also include a wireless communication module (such as a Bluetooth or WiFi system) for transmitting the data from the gas sensors to a remote location for analysis and treatment recommendations. A battery powers the negative pressure wound device and the gas sensors.

In addition to the above features, the negative pressure wound device may also include machine learning algorithms for analyzing the data from the gas sensors to predict the likelihood of infection and suggest treatment options. The device may also include a database for storing and organizing the data from the gas sensors, as well as a dashboard for displaying real-time data and providing alerts when the levels of gases indicative of infection are detected. The device may also include a reporting module for generating reports on the data from the gas sensors and the effectiveness of treatment interventions.

The negative pressure wound device may also include a display for displaying the results of the gas sensor readings. The display may be a visual display, such as an LCD screen, or may be an audio display, such as a speaker.

The negative pressure wound device may also include a transmitter for transmitting the gas sensor readings to a remote device for analysis and/or storage. The remote device may be a computer, smartphone, or other device with internet connectivity.

In use, the negative pressure wound device is applied to the wound and the pump is activated to apply negative pressure to the wound dressing. The gas sensors detect the gases present within the wound dressing and transmit the sensor readings to the display and/or the remote device. The results of the gas sensor readings may be used to determine the presence of wound infection and to prompt appropriate treatment.

The integration of gas sensors into a negative pressure wound device allows for the rapid and accurate detection of wound infection, improving patient outcomes and reducing the risk of complications. The device is easy to use and allows for continuous monitoring of the wound, enabling healthcare providers to promptly address any potential infection.

An embodiment of the present invention includes a system for the early detection and prediction of wound infections caused by bacteria and fungi. The system comprises a negative pressure wound device comprising a housing, a negative pressure source, and one or more gas sensors for the detection of gases produced by bacteria and fungi present in the wound. A control unit is configured to receive data from the gas sensors and to control the negative pressure source in response to the data. A user interface displays the data from the gas sensors, and a treatment module takes timely interventions to prevent or treat the infection based on the data from the gas sensors. A communication module transmits the data from the gas sensors to a remote location for analysis and treatment recommendations. Machine learning algorithms may be used for analyzing the data from the gas sensors to predict the likelihood of infection and suggest treatment (e.g., clean wound, change dressing, drugs, etc.).

An embodiment of the present invention includes a method for the early detection and prediction of wound infections caused by bacteria and fungi. A wound site is continuously monitored using one or more gas sensors integrated into a negative pressure wound device. Levels of specific gases produced by bacteria and fungi present in the wound are measured, and real-time data on the presence and levels of gases indicative of infection are provided to a control unit. The negative pressure applied by the negative pressure wound device is adjusted in response to the data from the gas sensors. The data from the gas sensors is displayed on a user interface. Timely interventions are taken to prevent or treat the infection based on the data from the gas sensors (e.g., clean wound, change dressing, drugs, etc.).

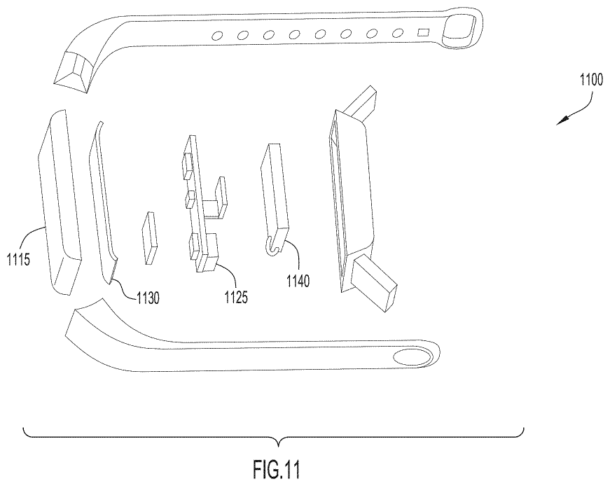

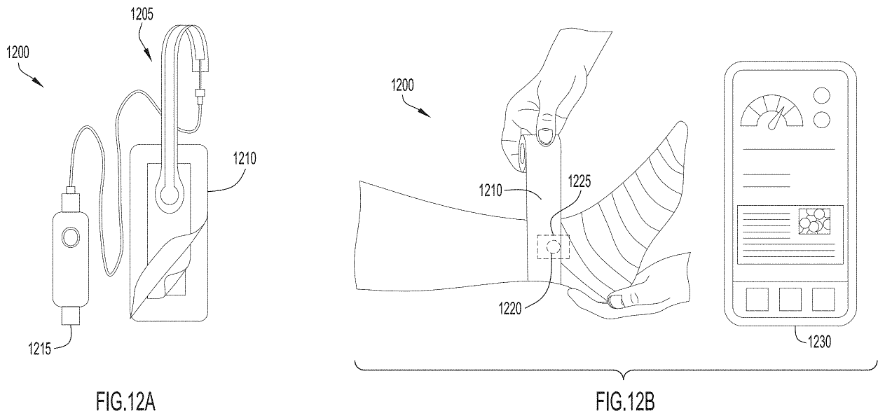

Referring to FIGS. 12A and 12B, a negative pressure wound device 1200 includes a housing 1205 with a wound dressing 1210 attached to the housing. The wound dressing is made of a porous material that allows for the passage of gases. A pump 1215 is in communication with the housing and is configured to apply negative pressure to the wound dressing. The pump may be a mechanical or electrical pump, and may be powered by a battery or other power source.

Gas sensors 1220 are positioned within the housing 1205 and are configured to detect gases present within the wound dressing 1210. The gas sensors may include sensors for detecting hydrogen and methane, which may be indicative of anaerobic bacteria present in the wound. The gas sensors may also include sensors for detecting other gases that may be indicative of wound infection. Housing 1205 is a container that houses the wound dressing 1210 and the gas sensors 1220. The gas sensors 1220 are positioned within the housing 1205, in close proximity to the wound dressing 1210. This allows the sensors to detect gases produced by bacteria and fungi in the wound and provide real-time data on the presence and levels of gases indicative of infection.

The gas sensors 1220 are in communication with a processor of the device, which is configured to process the sensor readings and transmit the results to a display and/or a transmitter of the device. The display may be a visual display, such as an LCD screen, or may be an audio display, such as a speaker. A user interface of the display can display real-time data on the levels of gases detected by the sensors, as well as other relevant information such as the type of bacteria or fungi present in the wound, and any recommended interventions or treatments.

The transmitter is configured to transmit the sensor readings to a remote device 1230 for analysis (determination of wound infection) and/or storage. The remote device may be a computer, smartphone, or other device with internet connectivity. The gas sensors and other device components may be substantially similar to the gas sensors and other components described above (FIGS. 1 and 2). A user interface of the remote device can display real-time data on the levels of gases detected by the sensors, as well as other relevant information such as the type of bacteria or fungi present in the wound, and any recommended interventions or treatments.

Based on collected information including VOCs patterns (and optionally vital signs and/or environment conditions), a machine learning algorithm such as Naive Bayes, Principal component analysis, Multinomial logistic regression, and Support-vector machines may be applied to train on the data and generate the correlations between certain infection issues with peak patterns (and optional pump adjustments and/or treatments). The interventions and treatments can be customized based on the specific needs of the patient and the type of infection present in the wound. For example, if the data from the gas sensors indicates the presence of anaerobic bacteria, the intervention might involve cleaning the wound and applying antibiotics that are effective against anaerobic bacteria. Alternatively, if the data suggests the presence of other types of bacteria or fungi, a different treatment plan may be recommended. The negative pressure wound device can be designed to adjust the level of negative pressure based on the levels of gases detected by the sensors. For example, if the gas sensors detect a higher level of hydrogen or methane gas, which may indicate the presence of anaerobic bacteria, the device can automatically increase the level of negative pressure to help remove the bacteria and promote wound healing.

Once the model is set up, with the VOC pattern (and optionally vital signs and/or environment conditions) as the input, the system can generate the early diagnosis result for wound infection purposes (and optionally a pump adjustment, intervening action, and/or treatment).

In use, negative pressure wound device 1200 is applied to the wound and the pump 1215 is activated to apply negative pressure to the wound dressing 1210. The gas sensors 1220 detect the gases present within the wound dressing (in substantially the same manner described above) and transmit the sensor readings to the processor. The processor processes the sensor readings in substantially the same manner described above, and transmits the results to the display and/or the transmitter. The results of the gas sensor readings may be displayed on the display and/or transmitted to the remote device 1230 for analysis and storage. The results may be used to determine the presence of wound infection and to prompt appropriate treatment. For example, the pump may be enabled in response to detection of an infection, a pumping rate adjusted in response to progression or remission of infection, etc. The negative wound pressure device with the gas sensors provides detections of bacteria similar to those shown in FIGS. 10A-10D described above.

The integration of gas sensors into a negative pressure wound device allows for the rapid and accurate detection of wound infection, improving patient outcomes and reducing the risk of complications. The device is easy to use and allows for continuous monitoring of the wound, enabling healthcare providers to promptly address any potential infection.

The gas sensors may be configured to detect other gases in addition to hydrogen, methane, carbon dioxide, ammonia and microbial Volatile organic compounds. The gas sensors may also be configured to detect multiple gases simultaneously or sequentially.

The negative pressure wound device may also include additional sensors or monitoring systems, such as temperature sensors or pH sensors, to provide additional information about the wound environment. The device may also include a user interface, such as buttons or a touch screen, to allow the user to adjust the negative pressure or other device settings.

The negative pressure wound device may also include a wireless or wired connection to a remote device, such as a computer or smartphone, for transmitting the gas sensor readings and other device data. The remote device may include software for analyzing the gas sensor readings and providing an indication of wound infection or other conditions. The remote device may also include a database for storing the gas sensor readings and other device data for future reference or analysis.

The negative pressure wound device may also include a power source, such as a battery or external power source, for powering the device and its components. The device may include a charging system, such as a USB port or charging port, for recharging the power source.

The negative pressure wound device may also include a casing or housing that is waterproof or water-resistant to protect the device and its components from moisture or other environmental factors. The casing or housing may also be sterilizable or disposable to reduce the risk of infection or contamination.

The negative pressure wound device may also include a timer or other tracking system to monitor the duration of treatment and to prompt the user to replace the wound dressing or other components as needed. The device may also include a memory or other storage system to store the gas sensor readings and other device data for future reference or analysis.

The negative pressure wound device may also include a user manual or other instructions to help the user properly operate and maintain the device. The instructions may include information on how to apply the device to the wound, how to activate and adjust the negative pressure, and how to interpret the gas sensor readings and other device data. The instructions may also include safety warnings and precautions to help the user avoid injury or damage to the device.

The negative pressure wound device allows for continuous monitoring of the wound, enabling healthcare providers to promptly address any potential infection and improve patient outcomes.

The negative pressure wound device may be designed and manufactured using a variety of materials and techniques to meet the needs and preferences of the user.

The wound dressing may be made of a porous and absorbent material, such as foam or gauze, to allow for the passage of gases and the absorption of exudate.

The gas sensors may be made of a sensitive and durable material, such as a semiconductor or metal oxide, to allow for accurate and reliable readings.

The pump may be made of a durable and reliable material, such as metal or plastic, to ensure long-lasting performance.

The negative pressure wound device may also be designed to be user-friendly and easy to operate, with clear and intuitive controls and displays. The device may be ergonomically designed to fit comfortably on the wound and to minimize the risk of discomfort or irritation to the user.

The negative pressure wound device may also be designed to be compatible with a variety of wound dressings and other accessories, such as adhesive patches or wraps, to allow for flexibility and customization. The device may also be designed to be compatible with a variety of power sources, such as batteries or external power sources, to allow for convenient and reliable operation.

In terms of manufacturing, the negative pressure wound device may be made using a variety of techniques, such as injection molding, blow molding, or extrusion, to create the desired shape and size. The device may also be subject to a variety of quality control measures to ensure that it meets the necessary performance and safety standards.

The negative pressure wound device may include, but not be limited to, a housing element composed of chip-systems, battery, IoT features, fan systems in various shapes, and port systems. In some embodiments, such devices will be, but not limited to, connected to sterile tubes, capillary systems, gas-permeable membranes, films, and cast systems. Such devices may include, but not limited to skin-safe adhesives, elastic bands, or a combination thereof to enable attachment to the anywhere on the human body.

In one embodiment of the negative pressure wound device, during operation, at least one of the one or more processors generates data by executing a method selected from Naive Bayes, principal component analysis (PCA), support vector machine (SVM), multi-layer perception (MLP), generalized regression neural network (GRNN), fuzzy inference systems (FIS), self-organizing map (SOM), radial bias function (RBF), genetic algorithms (GAS), neuro-fuzzy systems (NFS), adaptive resonance theory (ART), partial least squares (PLS), multiple linear regression (MLR), principal component regression (PCR), discriminant function analysis (DFA), linear discriminant analysis (LDA), cluster analysis, and nearest neighbor.

It will be appreciated that the embodiments described above and illustrated in the drawings represent only a few of the many ways of implementing embodiments for non-invasive detection of pathogens in wounds.

In some embodiments, detecting gas mixtures of pathogens comprises, at an operation temperature of 250° C. or less, exposing the gas mixtures to a sensor array comprising between 2 and 6 and 8 and 12 and 32 sensors or more with sensing materials, wherein the sensing material comprises at least one or two or more or mixture nanoporous structure, like mesoporous, macroporous, microporous, nanoporous, non-porous, hierarchical porous materials, including mesoporous/macroporous hierarchical structure, microporous/macroporous hierarchical structure, microporous/mesoporous hierarchical structure, microporous/mesoporous/macroporous hierarchical structure, etc. The mesoporous structure is in a configuration selected from a well-ordered mesoporous structure with regular pore arrangement, a worm-like mesoporous structure with uniform pore size but without long-range regularity, or a non-order mesostructure with pore size from 2-50 nm. The macroporous structure is a configuration selected from a well-ordered macroporous structure or a non-order macroporous structure with pore size from 50 nm to 50 μm. In a further embodiment, the pore size of sensing materials is ranging from 0.4 to 2 nm, 2-50 nm, 50 nm to 200 nm, 200 nm to 500 nm, 500 nm to 1 μm, 1-50 μm. The specific surface area is 1-1000 m²/g.

In some embodiments, the sensing material comprises unary, binary, ternary, quaternary, quinary, senary, septenary, and octonary multiple-component metal oxides, selected from the element group of tin (Sn), terbium (Tb), cobalt (Co), zinc (Zn), indium (In), copper (Cu), nickel (Ni), chromium (Cr), manganese (Mn), tungsten (W), titanium (Ti), vanadium (V), iron (Fe), aluminum (Al), gallium (Ga), silver (Ag), gold (Au), palladium (Pd), rhodium (Rh), ruthenium (Ru), molybdenum (Mo), niobium (Nb), zirconium (Zr), yttrium (Y), lanthanum (La), platinum (Pt), silicon (Si), cerium(Ce), tellurium (Te), such as CoZnInSnOx, CuSnCoInOx, CoZnCrNiOx, SnTbCoOx, SnTbZnOx, CoTbIn0x, CoNiTbOx, CoCeNiCuOx, ZnSnTeOx, CoZnInOx, CuSnInOx, CoCrNiOx, SnWLaOx, SnInLaCoOx, CoOx, CoTbOx with different compositions of each chemical element, x=0.01-1.

The set of sensor array signals detected in some embodiments may be obtained in response to the changes of electrical resistances of sensing materials.

The methods of some embodiments may comprise exposing the sensor array to gas mixtures emanated from pathogens of a wound infection. As used herein, gas mixtures may comprise VOCs or vapor from a subject, e.g., from the skin or breath of a subject. In some cases, the VOCs or vapor is emitted from the skin of a subject. The skin may be that of any wound part of the subject, e.g., the palm, finger, arm, leg, back, abdomen, or foot of the subject. In some examples, the gas mixtures comprise diverse odor of chemical classes, such as aldehydes, alcohols, ketones, acids, Sulphur containing compounds, esters, hydrocarbons and nitrogen containing compounds.

The methods of some embodiments may be performed at a relatively low operation temperature. In some embodiments, the operation temperature may be at most 250° C., at most 2000° C., at most 1500° C., at most 1000° C., at most 80° C., at most 60° C., at most 50° C., at most 40° C., at most 30° C., at most 20° C., or at most 10° C. In some examples, the operation temperature may be in a range from about −30° C. to about 40° C., e.g., from about 0° C. to 30° C., from about 10° C. to about 30° C., or from about 20° C. to about 25° C. In some examples, the operation temperature may be 50° C., or less.

The methods, gas sensors, and devices of some embodiments may detect relatively levels of gas mixtures. In some cases, the methods and devices of some embodiments may be capable of detecting VOCs at a concentration of 5000 parts per million (ppm) or less, 4000 ppm or less, 3000 ppm or less, 2000 ppm or less, 1000 ppm or less, 500 ppm or less, 250 ppm or less, 100 ppm or less, 50 ppm or less, 10 ppm or less, 1 ppm or less, 800 parts per billion (ppb) or less, 600 ppb or less, 500 ppb or less, 400 ppb or less, 200 ppb or less, 100 ppb or less, 80 ppb or less, 60 ppb or less, 40 ppb or less, 20 ppb or less, 10 ppb or less, or 1 ppb or less, of gases in gas mixtures. In some cases, the methods, sensors, and devices of some embodiments may be configured to have a limit of detection of 5000 ppm or less of gases in gas mixtures. By “limit of detection” is meant the lowest quantity of a substance that can be distinguished from the absence of that substance (e.g., a blank value). In certain cases, the gas sensor or device of some embodiments are configured to have a limit of detection of 1000 ppm or less, 500 ppm or less, such as 400 ppm or less, including 300 ppm or less, 200 ppm or less, 100 ppm or less, 75 ppm or less, 50 ppm or less, 25 ppm or less, 20 ppm or less, 15 ppm or less, 10 ppm or less, 5 ppm or less, 1 ppm or less, 500 ppb or less, 100 ppb or less, 50 ppb or less, 10 ppb or less, or 1 ppb or less. In certain cases, the gas sensor or device of some embodiments is configured to have a limit of detection of 1 ppm or less. In certain cases, the gas sensor or device is configured to detect at least 1 ppb, at least 10 ppb, at least 50 ppb, at least 100 ppb, at least 500 ppb, at least 1 ppm, at least 5 ppm, at least 10 ppm, at least 15, ppm, at least 20 ppm, at least 25 ppm, at least 50 ppm, at least 75 ppm, at least 100 ppm, or at least 200 ppm of the VOCs.

In some embodiments, the sensor array may detect both the gas mixtures having the volatile compounds contained or accumulated therein and gas mixtures not having the volatile compounds contained or accumulated therein.

The subject may relate to an animal, including mammals, preferably humans, to which the method of some embodiments may be applied. Mammalian species that can benefit from embodiments of the invention include, but are not limited to: apes, chimpanzees, orangutans, humans, monkeys; domesticated animals (e.g. pets) such as dogs, cats, guinea pigs, hamsters; and large domesticated animals such as cattle, horses, goats, sheep. Also, a subject could be any wild animal, as embodiments of the present invention could be used for tracking purposes.

In some embodiments, identification of bacteria may be based on volatile organic or inorganic compounds obtained from pathogens of a wound infection in a subject with a sensor array. A sensor array may be any device capable of generating an electrical signal changing in response to interaction with volatile compounds of interest. The sensor signals may be any observable change in one or more quantifiable entities such as resistance, voltage, frequency, and the like.

In some embodiments, the reference signals comprise known pathogens, such as bacteria and fungi. The reference signals could be established using standard samples obtained from in vitro grown pathogens, or in vivo infected and non-infected patients or animals that are assessed by embodiments of the present invention and/or by conventional techniques to identify pathogens found therein.

The data acquisition unit may comprise nanosensor(s), biosensor(s), read-out circuit(s), one or more microcontrollers (e.g., signal converter, signal processing, control circuit, power control integrated circuit(s), etc.), a communication unit, and a battery.

In some embodiments, the device of some embodiments includes one or more of the following features or attributes: skin and body temperature (−15° C. to 45° C.), heart rate, humidity (0-99%), concentration of volatile organic compounds (1 ppb-5000 ppm), provide an audible alarm and inaudible alarm or color alert or other visualization or notification when the pathogens are detected, can be directly integrated into existing products (e.g. wound dressing system, wound healing system, wound management system).

Devices of some embodiments comprise one or more of the gas sensors described above. In certain embodiments, the devices further comprise a power supply, display, a computer, a microcontroller unit, a read-out circuit, a communication module (e.g., a wireless communication module), a memory, or any combination thereof. The gas sensors and/or devices may detect and differentiate two or more pathogens (e.g., bacteria, fungi, etc.) in polymicrobial infections based on change patterns of sensing material properties.

The devices of some embodiments may be stand-alone, and/or incorporated in (e.g., as a part of) and/or interoperable with interactive mobile devices or applications with Internet of Things (IoT) features. In some embodiments, the devices may be integrated to or a part of professional wound dressing systems, hypothermia bag, transport chamber, smartphones, wearable devices, health care devices, medical devices, fitness equipment (e.g., treadmill, elliptical, etc.), or a combination thereof. The device may detect VOCs, e.g., those from breath or emitted from the skin (e.g., the skin of, the palm, finger, ear, nose, face, eye, arm, leg, chest, breast, back, abdomen, or foot of a subject).

The devices of some embodiments may be wearable devices. In some cases, with the sensing materials herein, a gas sensor array sensitive to the pathogen of wound infection emitted VOCs may be fabricated as a wearable device. Examples of wearable devices include an armband, a sleeve, a jacket, glasses, eye wears, goggles, a glove, a watch, a wristband, a bracelet, ear bud, earphone, an article of clothing, a hat, a headband, a headset, a bra, and jewelry.

The devices of some embodiments may be portable devices. In some cases, with the sensing materials herein, a gas sensor array sensitive to the VOCs emanated from pathogens of a wound infection may be fabricated as a portable device by deposition. Examples of portable devices include a keychain, a Breathalyzer, etc.

The devices of some embodiments may be disposable devices (e.g., wound dressing, etc.), where the disposable device (and the one or more sensors) are configured for disposable use and may be replaced after each use.

The devices of some embodiments may be functional with relatively low power consumption. For example, the devices of some embodiments may have a power consumption of at most 500 μAmp, at most 400 μAmp, at most 300 μAmp, at most 200 μAmp, at most 20 μAmp, at most 10 μAmp, at most 9 μAmp, at most 8 μAmp, at most 7 μAmp, at most 6 μAmp, at most 5 μAmp, at most 4 μAmp, at most 3 μAmp, at most 2 μAmp, or at most 1 μAmp.

The devices of some embodiments may be relatively small in size. For example, the device may have a volume of at most 300 cm³, at most 200 cm³, at most 100 cm³, at most 30 cm³, at most 20 cm³, at most 15 cm³, at most 10 cm³, at most 8 cm³, at most 6 cm³, at most 5 cm³, at most 4 cm³, at most 3 cm³, at most 2 cm³, or at most 1 cm³.

In some cases, the devices of some embodiments are intelligent. For example, the devices may be configured to calibrate (e.g., self-calibrate). The calibration may be performed based on reference information specific for an individual user.

The devices of some embodiments may be configured to digitally read VOCs concentrations. The devices may convert signals from one form to another. For example, the devices may convert analog signals into digital signals, and/or convert digital signals into measurements of energy consumption and/or metabolic profiles of the user subject.

The devices of some embodiments may transfer data wirelessly, e.g., via internet, Bluetooth, Bluetooth low energy (BLE), or a combination thereof. The devices may be configured to connect with smartphones or computers (e.g., laptops) to visualize, monitor, and/or analyze the infection development and progression, antibiotic treatment, metabolic profiles and physiological statuses, or a combination thereof of a subject using (e.g., wearing) the devices.

Data of a device of some embodiments can be shared with medical professionals in real-time to realize more accurate and appropriate treatment.

The devices (e.g., FIGS. 1, 6A, 6B, 8, 11, 12A, and 12B) may determine intervening actions and/or treatments as described above. Based on collected information including VOCs patterns (and optionally vital signs and/or environment conditions), a machine learning algorithm such as Naive Bayes, Principal component analysis, Multinomial logistic regression, and Support-vector machines may be applied in embodiments to train on the data and generate the correlations between certain infection issues with peak patterns (and optional intervening actions, pressure source controls, and/or treatments). Once the model is set up, with the VOC pattern (and optionally vital signs and/or environment conditions) as the input, the system can generate the early diagnosis result for wound infection purposes (and optionally a pressure source adjustment, an intervening action, and/or a treatment).

The device of some embodiments may sense the presence of wound infection continuously. For example, the device of some embodiments may monitor pathogen growth in real-time from incubation, through colonization, and until infection.

In some embodiments, the one or more pathogens include at least one from a group of bacteria and fungi. The bacteria may include Escherichia coli, Salmonella enterica, Staphylococcus aureus, Streptococcus pneumoniae, Streptococcus pyogenes, Neisseria gonorrhoeae, Neisseria meningitidis, Haemophilus influenzae, Pseudomonas aeruginosa, Klebsiella pneumoniae, Enterococcus faecalis, Enterococcus faecium, Clostridioides difficile, Campylobacter jejuni, Listeria monocytogenes, Vibrio cholerae, Vibrio parahaemolyticus, Mycobacterium tuberculosis, Mycobacterium leprae, Helicobacter pylori, Bordetella pertussis, Legionella pneumophila, Shigella spp., Yersinia pestis, Francisella tularensis, Brucella spp., Borrelia burgdorferi, Chlamydia trachomatis, Chlamydia pneumoniae, Coxiella burnetiid, Rickettsia rickettsia, Rickettsia prowazekii, Bartonella henselae, Burkholderia pseudomallei, Burkholderia mallei, Acinetobacter baumannii, Moraxella catarrhalis, Nocardia spp., Propionibacterium acnes, Actinomyces spp., Treponema pallidum, Treponema denticola, Fusobacterium spp., Porphyromonas spp., Prevotella spp., Bacteroides fragilis, Bacteroides thetaiotaomicron, Capnocytophaga spp., Pasteurella multocida, Actinobacillus spp., Streptobacillus moniliformis, Erysipelothrix rhusiopathiae, Lactobacillus spp., Corynebacterium diphtheriae, Corynebacterium jeikeium, Nocardia asteroids, Mycoplasma pneumoniae, Ureaplasma urealyticum, Legionella longbeachae, Legionella bozemanii, Legionella dumoffii, Legionella micdadei, Legionella anisa, Legionella feeleii, Legionella gormanii, Legionella jordanis, Legionella londiniensis, Legionella maceachernii, Legionella oakridgensis, Legionella quateirensis, Legionella rubrilucens, Legionella sainthelensi, Legionella steigerwaltii, Legionella taurinensis, and Legionella wadsworthii.

The fungi may include Candida albicans, Aspergillus fumigatus, Cryptococcus neoformans, Histoplasma capsulatum, Blastomyces dermatitidis, Coccidioides immitis, Candida glabrata, Candida tropicalis, Candida parapsilosis, Candida krusei, Trichophyton rubrum, Trichophyton mentagrophytes, Microsporum canis, Epidermophyton floccosum, Pneumocystis jirovecii, Fusarium solani, Fusarium oxysporum, Rhizopus oryzae, Mucor spp., Scedosporium prolificans, Sporothrix schenckii, Paracoccidioides brasiliensis, Candida dubliniensis, Candida lusitaniae, Candida guilliermondii, Candida kefyr, Candida famata, Candida lipolytica, Candida utilis, Candida zeylanoides, Candida rugosa, Candida norvegensis, Candida pelliculosa, Candida sake Candida stellatoidea, Candida zonata, Aspergillus flavus, Aspergillus niger, Aspergillus terreus, Candida haemulonii, Candida orthopsilosis, Candida metapsilosis, Candida auris, Trichosporon asahii, Trichosporon cutaneum, Trichosporon mucoides, Trichosporon ovoides, Trichosporon asteroid, Geotrichum candidum, Geotrichum capitatum, Paecilomyces spp., Acremonium spp., Alternaria spp., Cladosporium spp., Penicillium spp., Aspergillus nidulans, Aspergillus versicolor, Exophiala dermatitidis, Exophiala jeanselmei, Exophiala spinifera, Exophiala xenobiotica, Candida utilis var. utilis, Candida glabrata var. bracarensis, Trichosporon dohaense, Trichosporon domesticum, Trichosporon japonicum, Trichosporon moniliiforme, Trichosporon mucoidum, Trichosporon pullulans, Rhizomucor pusillus, Rhizomucor variabilis, Cunninghamella bertholletiae, Cunninghamella echinulate, Cunninghamella blakesleeana, Absidia corymbifera, Mucor circinelloides, Mucor racemosus, Saksenaea vasiformis, Rhizopus microspores, and Rhizopus spp.

Various modifications and variations of the described embodiments will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out embodiments of the invention that are obvious to those skilled in the art are intended to be within the scope of the invention. This application is intended to cover any variations, uses, or adaptations of embodiments of the invention following, in general, the principles of the invention and including such departures from the present disclosure that come within known customary practice within the art to which the invention pertains and may be applied to the essential features herein before set forth.

What is claimed is:

1. A method of detecting a wound infection comprising:
receiving, from at least one sensor, information pertaining to detection of one or more gases emanating from one or more pathogens in a wound that produce an infection, wherein the at least one sensor includes sensing materials that change one or more properties in response to a presence of the one or more gases; and
analyzing, via at least one processor, the information from the at least one sensor to identify the one or more pathogens and determine a presence of the infection in the wound, wherein the one or more pathogens are identified based on patterns of changes of the one or more properties indicating corresponding pathogens.

2. The method of claim 1, wherein the at least one sensor further provides measurements of one or more from a group of physiological parameters and environment conditions, and analyzing the information comprises:
analyzing the information and measurements from the at least one sensor to identify the one or more pathogens and determine the presence of the infection in the wound, wherein the one or more pathogens are identified based on patterns of changes of the one or more properties and the measurements indicating the corresponding pathogens.

3. The method of claim 1, wherein the one or more pathogens include two or more pathogens from a group of bacteria and fungi, and the method further comprises:
detecting and differentiating the two or more pathogens in polymicrobial infections.

4. The method of claim 1, wherein analyzing the information comprises:
analyzing the information by a machine learning model to correlate the patterns of changes of the one or more properties to patterns of the corresponding pathogens.

5. The method of claim 1, wherein the at least one sensor is disposed within one of a wearable device, a portable device, a disposable device, and a wound dressing, and the method further comprises:
monitoring pathogen growth in real-time from incubation, colonization, until infection.

6. The method of claim 1, wherein the at least one sensor is further configured to differentiate two or more pathogens in polymicrobial infections.

7. The method of claim 1, wherein the information from the at least one sensor is monitored in real-time.

8. The method of claim 1, wherein the at least one sensor is disposed within a wound dressing, and the wound dressing and the at least one sensor are configured for disposable use.

9. The method of claim 1, wherein the one or more properties of the sensing materials that change include electrical conductivity, capacitance, resistance, or impedance.

10. The method of claim 1, further comprising providing alerts or notifications to healthcare providers or patients based on the information from the at least one sensor.

11. The method of claim 1, wherein the one or more pathogens include at least one from a group of bacteria and fungi, and wherein analyzing further comprises identifying the one or more pathogens based on patterns of changes of the one or more properties corresponding to the bacteria and fungi.

12. The method of claim 1, wherein the one or more pathogens include at least one from a group of bacteria and fungi, wherein the bacteria include Escherichia coli, Salmonella enterica, Staphylococcus aureus, Streptococcus pneumoniae, Streptococcus pyogenes, Neisseria gonorrhoeae, Neisseria meningitidis, Haemophilus influenzae, Pseudomonas aeruginosa, Klebsiella pneumoniae, Enterococcus faecalis, Enterococcus faecium, Clostridioides difficile, Campylobacter jejuni, Listeria monocytogenes, Vibrio cholerae, Vibrio parahaemolyticus, Mycobacterium tuberculosis, Mycobacterium leprae, Helicobacter pylori, Bordetella pertussis, Legionella pneumophila, Shigella spp., Yersinia pestis, Francisella tularensis, Brucella spp., Borrelia burgdorferi, Chlamydia trachomatis, Chlamydia pneumoniae, Coxiella burnetiid, Rickettsia rickettsia, Rickettsia prowazekii, Bartonella henselae, Burkholderia pseudomallei, Burkholderia mallei, Acinetobacter baumannii, Moraxella catarrhalis, Nocardia spp., Propionibacterium acnes, Actinomyces spp., Treponema pallidum, Treponema denticola, Fusobacterium spp., Porphyromonas spp., Prevotella spp., Bacteroides fragilis, Bacteroides thetaiotaomicron, Capnocytophaga spp., Pasteurella multocida, Actinobacillus spp., Streptobacillus moniliformis, Erysipelothrix rhusiopathiae, Lactobacillus spp., Corynebacterium diphtheriae, Corynebacterium jeikeium, Nocardia asteroids, Mycoplasma pneumoniae, Ureaplasma urealyticum, Legionella longbeachae, Legionella bozemanii, Legionella dumoffii, Legionella micdadei, Legionella anisa, Legionella feeleii, Legionella gormanii, Legionella jordanis, Legionella londiniensis, Legionella maceachernii, Legionella oakridgensis, Legionella quateirensis, Legionella rubrilucens, Legionella sainthelensi, Legionella steigerwaltii, Legionella taurinensis, and Legionella wadsworthii, and the fungi include Candida albicans, Aspergillus fumigatus, Cryptococcus neoformans, Histoplasma capsulatum, Blastomyces dermatitidis, Coccidioides immitis, Candida glabrata, Candida tropicalis, Candida parapsilosis, Candida krusei, Trichophyton rubrum, Trichophyton mentagrophytes, Microsporum canis, Epidermophyton floccosum, Pneumocystis jirovecii, Fusarium solani, Fusarium oxysporum, Rhizopus oryzae, Mucor spp., Scedosporium prolificans, Sporothrix schenckii, Paracoccidioides brasiliensis, Candida dubliniensis, Candida lusitaniae, Candida guilliermondii, Candida kefyr, Candida famata, Candida lipolytica, Candida utilis, Candida zeylanoides, Candida rugosa, Candida norvegensis, Candida pelliculosa, Candida sake Candida stellatoidea, Candida zonata, Aspergillus flavus, Aspergillus niger, Aspergillus terreus, Candida haemulonii, Candida orthopsilosis, Candida metapsilosis, Candida auris, Trichosporon asahii, Trichosporon cutaneum, Trichosporon mucoides, Trichosporon ovoides, Trichosporon asteroid, Geotrichum candidum, Geotrichum capitatum, Paecilomyces spp., Acremonium spp., Alternaria spp., Cladosporium spp., Penicillium spp., Aspergillus nidulans, Aspergillus versicolor, Exophiala dermatitidis, Exophiala jeanselmei, Exophiala spinifera, Exophiala xenobiotica, Candida utilis var. utilis, Candida glabrata var. bracarensis, Trichosporon dohaense, Trichosporon domesticum, Trichosporon japonicum, Trichosporon moniliiforme, Trichosporon mucoidum, Trichosporon pullulans, Rhizomucor pusillus, Rhizomucor variabilis, Cunninghamella bertholletiae, Cunninghamella echinulate, Cunninghamella blakesleeana, Absidia corymbifera, Mucor circinelloides, Mucor racemosus, Saksenaea vasiformis, Rhizopus microspores, and Rhizopus spp.

13. The method of claim 1, wherein the at least one sensor is disposed within a wound dressing, and the method further comprises:
applying negative pressure to the wound, via a negative pressure source, to promote healing.

14. The method of claim 13, further comprising:
adjusting a rate of the negative pressure source based on the information from the at least one sensor.

15. The method of claim 1, further comprising:
determining, via the at least one processor, a treatment for the wound based on the information from the at least one sensor.

16. A system for detecting a wound infection comprising:
at least one sensor to detect one or more gases emanating from one or more pathogens in a wound that produce an infection, wherein the at least one sensor includes sensing materials that change one or more properties in response to a presence of the one or more gases; and
at least one processor configured to:
analyze information from the at least one sensor to identify the one or more pathogens and determine a presence of the infection in the wound, wherein the one or more pathogens are identified based on patterns of changes of the one or more properties indicating corresponding pathogens.

17. The system of claim 16, wherein the at least one sensor further provides measurements of one or more from a group of physiological parameters and environment conditions, and analyzing the information comprises:
analyzing the information and measurements from the at least one sensor to identify the one or more pathogens and determine the presence of the infection in the wound, wherein the one or more pathogens are identified based on patterns of changes of the one or more properties and the measurements indicating the corresponding pathogens.

18. The system of claim 16, wherein the one or more pathogens include at least one from a group of bacteria and fungi, wherein the bacteria include Escherichia coli, Salmonella enterica, Staphylococcus aureus, Streptococcus pneumoniae, Streptococcus pyogenes, Neisseria gonorrhoeae, Neisseria meningitidis, Haemophilus influenzae, Pseudomonas aeruginosa, Klebsiella pneumoniae, Enterococcus faecalis, Enterococcus faecium, Clostridioides difficile, Campylobacter jejuni, Listeria monocytogenes, Vibrio cholerae, Vibrio parahaemolyticus, Mycobacterium tuberculosis, Mycobacterium leprae, Helicobacter pylori, Bordetella pertussis, Legionella pneumophila, Shigella spp., Yersinia pestis, Francisella tularensis, Brucella spp., Borrelia burgdorferi, Chlamydia trachomatis, Chlamydia pneumoniae, Coxiella burnetiid, Rickettsia rickettsia, Rickettsia prowazekii, Bartonella henselae, Burkholderia pseudomallei, Burkholderia mallei, Acinetobacter baumannii, Moraxella catarrhalis, Nocardia spp., Propionibacterium acnes, Actinomyces spp., Treponema pallidum, Treponema denticola, Fusobacterium spp., Porphyromonas spp., Prevotella spp., Bacteroides fragilis, Bacteroides thetaiotaomicron, Capnocytophaga spp., Pasteurella multocida, Actinobacillus spp., Streptobacillus moniliformis, Erysipelothrix rhusiopathiae, Lactobacillus spp., Corynebacterium diphtheriae, Corynebacterium jeikeium, Nocardia asteroids, Mycoplasma pneumoniae, Ureaplasma urealyticum, Legionella longbeachae, Legionella bozemanii, Legionella dumoffii, Legionella micdadei, Legionella anisa, Legionella feeleii, Legionella gormanii, Legionella jordanis, Legionella londiniensis, Legionella maceachernii, Legionella oakridgensis, Legionella quateirensis, Legionella rubrilucens, Legionella sainthelensi, Legionella steigerwaltii, Legionella taurinensis, and Legionella wadsworthii, and the fungi include Candida albicans, Aspergillus fumigatus, Cryptococcus neoformans, Histoplasma capsulatum, Blastomyces dermatitidis, Coccidioides immitis, Candida glabrata, Candida tropicalis, Candida parapsilosis, Candida krusei, Trichophyton rubrum, Trichophyton mentagrophytes, Microsporum canis, Epidermophyton floccosum, Pneumocystis jirovecii, Fusarium solani, Fusarium oxysporum, Rhizopus oryzae, Mucor spp., Scedosporium prolificans, Sporothrix schenckii, Paracoccidioides brasiliensis, Candida dubliniensis, Candida lusitaniae, Candida guilliermondii, Candida kefyr, Candida famata, Candida lipolytica, Candida utilis, Candida zeylanoides, Candida rugosa, Candida norvegensis, Candida pelliculosa, Candida sake Candida stellatoidea, Candida zonata, Aspergillus flavus, Aspergillus niger, Aspergillus terreus, Candida haemulonii, Candida orthopsilosis, Candida metapsilosis, Candida auris, Trichosporon asahii, Trichosporon cutaneum, Trichosporon mucoides, Trichosporon ovoides, Trichosporon asteroid, Geotrichum candidum, Geotrichum capitatum, Paecilomyces spp., Acremonium spp., Alternaria spp., Cladosporium spp., Penicillium spp., Aspergillus nidulans, Aspergillus versicolor, Exophiala dermatitidis, Exophiala jeanselmei, Exophiala spinifera, Exophiala xenobiotica, Candida utilis var. utilis, Candida glabrata var. bracarensis, Trichosporon dohaense, Trichosporon domesticum, Trichosporon japonicum, Trichosporon moniliiforme, Trichosporon mucoidum, Trichosporon pullulans, Rhizomucor pusillus, Rhizomucor variabilis, Cunninghamella bertholletiae, Cunninghamella echinulate, Cunninghamella blakesleeana, Absidia corymbifera, Mucor circinelloides, Mucor racemosus, Saksenaea vasiformis, Rhizopus microspores, and Rhizopus spp.

19. The system of claim 16, wherein analyzing the information comprises:
analyzing the information by a machine learning model to correlate the patterns of changes of the one or more properties to patterns of the corresponding pathogens.

20. The system of claim 16, wherein the at least one sensor is disposed within one of a wearable device, a portable device, a disposable device, and a wound dressing.

21. The system of claim 20, wherein the at least one processor is disposed in a remote device.

22. The system of claim 20, wherein the at least one sensor is disposed within the wound dressing, and the system further comprises:
a negative pressure source to apply negative pressure to the wound to promote healing.

23. The system of claim 22, wherein the at least one processor is further configured to:
adjust a rate of the negative pressure source based on the information from the at least one sensor.

24. The system of claim 16, wherein the at least one processor is further configured to:
determine a treatment for the wound based on the information from the at least one sensor.

25. An apparatus comprising:
a memory device containing software executable by at least one processor to cause the at least one processor to:
receive, from at least one sensor, information pertaining to detection of one or more gases emanating from one or more pathogens in a wound that produce an infection, wherein the at least one sensor includes sensing materials that change one or more properties in response to a presence of the one or more gases; and
analyze the information from the at least one sensor to identify the one or more pathogens and determine a presence of the infection in the wound, wherein the one or more pathogens are identified based on patterns of changes of the one or more properties indicating corresponding pathogens.

26. The apparatus of claim 25, wherein the one or more pathogens include at least one from a group of bacteria and fungi, and wherein analyzing the information comprises:
analyzing the information by a machine learning model to correlate the patterns of changes of the one or more properties to patterns of the corresponding pathogens.

27. The apparatus of claim 25, wherein a negative pressure source applies negative pressure to the wound to promote healing, and the software further causes the at least one processor to:
adjust a rate of the negative pressure source based on the information from the at least one sensor.

28. The apparatus of claim 25, wherein the software further causes the at least one processor to:
determine a treatment for the wound based on the information from the at least one sensor.
